(19) United States
(12) Patent Application Publication
(10) Pub. No.: US 2021/0009650 A1
(43) Pub. Date: Jan. 14, 2021

(54) MODIFIED FIBROBLAST GROWTH FACTORS AND USES THEREOF

(71) Applicant: Trefoil Therapeutics, Inc., San Diego, CA (US)

(72) Inventors: David EVELETH, San Diego, CA (US); Ralph A. BRADSHAW, Encinitas, CA (US)

(21) Appl. No.: 16/089,698

(22) PCT Filed: Mar. 24, 2017
(86) PCT No.: PCT/US2017/024048

§ 371 Date: Sep. 28, 2018

Related U.S. Application Data
(60) Provisional application No. 62/314,101, filed on Mar. 28, 2016.

(51) Int. Cl. C07K 14/50; A61K 47/69; A61P 27/02

(52) U.S. Cl. CPC C07K 14/50; A61P 27/02; A61K 47/6929

(57) ABSTRACT

Described herein are modified fibroblast growth factors (FGFs), pharmaceutical compositions, formulations, and medicaments that include such modified FGFs, and methods of using such modified FGFs to treat mammalian diseases, disorders, or conditions.

### CROSS-REFERENCE

This application claims the benefit of U.S. Provisional Application No. 62/314,101, filed Mar. 28, 2016, which is incorporated herein by reference in its entirety.

### FIELD OF THE INVENTION

Described herein are modified fibroblast growth factors (FGFs), pharmaceutical compositions, and medicaments that include such modified FGFs, kits that include such modified FGFs, and methods of using such modified FGFs to treat diseases, disorders, or conditions.

### BACKGROUND OF THE INVENTION

Several members of the FGF family of proteins have the potential of providing therapy for the treatment of one or more diseases, disorders, or conditions. For example, one or more members of the FGF family of proteins are potentially useful to treat a wound, for example a skin wound, a broken bone, a bone lesion, and/or rejuvenate or reduce scar formation. Additionally, several members of the FGF family of proteins, including FGF-1, have the potential of providing “angiogenic therapy” for the treatment of ischemic conditions or diseases (i.e., diseases caused by insufficient blood flow to one or more tissues), such as coronary artery disease, peripheral vascular disease, peripheral arterial occlusion or disease (e.g., critical limb ischemia or CLI), etc., by triggering neovascularization of affected tissues. See, e.g., Nikol, S. et al., “Therapeutic Angiogenesis With Intramuscular NV1FGF Improves Amputation-free Survival in Patients With Critical Limb Ischemia,” Mol Ther 16(5):972-978 (2008).

### SUMMARY OF THE INVENTION

In some aspects the compositions, formulations, medicaments, and methods to treat mammalian diseases, disorders, or conditions provided herein comprise a modified FGF, the modified FGF comprising one or more mutations of human wild type FGF-1 (SEQ ID NO: 109) at positions K9, K10, P11, K12, C16, C83, C117, P134, P136, and V137. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects the compositions, formulations, medicaments, and methods to treat mammalian diseases, disorders, or conditions provided herein comprise a modified FGF, a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects, provided herein are methods of treating an injury, disease, or condition in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating or preventing an ocular disease disorder or condition in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the pharmaceutical composition is free of heparin. In some embodiments, the pharmaceutical composition is a liquid ophthalmic formulation. In some embodiments, the ophthalmic formulation is administered topically, by microneedle into the cornea, or intracamerally. In some embodiments, the ophthalmic formulation is administered by an eye drop. In some embodiments, the ocular disease, disorder or condition is a disease, disorder, or condition of the cornea, ocular surface, corneal endothelium, or corneal epithelium. In some embodiments, the disease, disorder, or condition of the corneal endothelium is Fuch's dystrophy, bullous keratopathy, congenital hereditary endothelial dystrophy 1, congenital hereditary endothelial dystrophy 2, posterior polymorphous corneal dystrophy, or a dry eye syndrome. In some embodiments, the ocular disease, disorder or condition is Fuch's dystrophy. In some embodiments, the condition of the corneal epithelium is a dry eye syndrome or corneal epithelial damage from corneal surgery or transplantation. In some embodiments, the corneal surgery is photorefractive keratotomy (PRK) or laser-assisted in situ keratomileusis (LASIK). In some embodiments, the ocular disease, disorder or condition is a disease, disorder, or condition of the corneal stroma. In some embodiments, the disease, disorder, or condition of the corneal stroma is keratoconus, lattice corneal dystrophy, granular corneal dystrophy, macular corneal dystrophy, Schnyder crystalline corneal dystrophy, congenital stromal corneal dystrophy, or fleck corneal dystrophy. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects, provided herein are methods of treating a corneal or retinal disease in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the pharmaceutical composition is free of heparin. In some embodiments, the pharmaceutical composition is a liquid ophthalmic formulation. In some embodiments, the ophthalmic formulation is administered topically, by microneedle into the cornea, or intracamerally. In some embodiments, the ophthalmic formulation is administered by an eye drop. In some embodiments, the ocular disease, disorder or condition is a disease, disorder, or condition of the cornea, ocular surface, corneal endothelium, or corneal epithelium. In some embodiments, the disease, disorder, or condition of the corneal endothelium is Fuch's dystrophy, bullous keratopathy, congenital hereditary endothelial dystrophy 1, congenital hereditary endothelial dystrophy 2, posterior polymorphous corneal dystrophy, or a dry eye syndrome. In some embodiments, the ocular disease, disorder or condition is Fuch's dystrophy. In some embodiments, the condition of the corneal epithelium is a dry eye syndrome or corneal epithelial damage from corneal surgery or transplantation. In some embodiments, the corneal surgery is photorefractive keratotomy (PRK) or laser-assisted in situ keratomileusis (LASIK). In some embodiments, the ocular disease, disorder or condition is a disease, disorder, or condition of the corneal stroma. In some embodiments, the disease, disorder, or condition of the corneal stroma is keratoconus, lattice corneal dystrophy, granular corneal dystrophy, macular corneal dystrophy, Schnyder crystalline corneal dystrophy, congenital stromal corneal dystrophy, or fleck corneal dystrophy. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects, provided herein are methods of treating a wound in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating a broken bone in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating a bone lesion in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of rejuvenating or reducing scar formation in a mammal comprising administering to the mammal a pharmaceutical composition comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating an injury, disease, or condition in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating or preventing an ocular disease disorder or condition in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the pharmaceutical composition is free of heparin. In some embodiments, the pharmaceutical composition is a liquid ophthalmic formulation. In some embodiments, the ophthalmic formulation is administered topically, by microneedle into the cornea, or intracamerally. In some embodiments, the ophthalmic formulation is administered by an eye drop. In some embodiments, the ocular disease, disorder or condition is a disease, disorder, or condition of the cornea, ocular surface, the corneal endothelium, or the corneal epithelium. In some embodiments, the disease, disorder, or condition of the corneal endothelium is Fuch's dystrophy, bullous keratopathy, congenital hereditary endothelial dystrophy 1, congenital hereditary endothelial dystrophy 2, posterior polymorphous corneal dystrophy, or a dry eye syndrome. In some embodiments, the ocular disease, disorder or condition is Fuch's dystrophy. In some embodiments, the condition of the corneal epithelium is a dry eye syndrome or corneal epithelial damage from corneal surgery or transplantation. In some embodiments, the corneal surgery is photorefractive keratotomy (PRK) or laser-assisted in situ keratomileusis (LASIK). In some embodiments, the ocular disease, disorder or condition is a disease, disorder, or condition of the corneal stroma. In some embodiments, the disease, disorder, or condition of the corneal stroma is keratoconus, lattice corneal dystrophy, granular corneal dystrophy, macular corneal dystrophy, Schnyder crystalline corneal dystrophy, congenital stromal corneal dystrophy, or fleck corneal dystrophy. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects, provided herein are methods of treating a corneal or retinal disease in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the pharmaceutical composition is free of heparin. In some embodiments, the pharmaceutical composition is a liquid ophthalmic formulation. In some embodiments, the ophthalmic formulation is administered topically, by microneedle into the cornea, or intracamerally. In some embodiments, the ophthalmic formulation is administered by an eye drop. In some embodiments, the ocular disease, disorder or condition is a disease, disorder, or condition of the cornea, ocular surface, the corneal endothelium, or the corneal epithelium. In some embodiments, the disease, disorder, or condition of the corneal endothelium is Fuch's dystrophy, bullous keratopathy, congenital hereditary endothelial dystrophy 1, congenital hereditary endothelial dystrophy 2, posterior polymorphous corneal dystrophy, or a dry eye syndrome. In some embodiments, the ocular disease, disorder or condition is Fuch's dystrophy. In some embodiments, the condition of the corneal epithelium is a dry eye syndrome or corneal epithelial damage from corneal surgery or transplantation. In some embodiments, the corneal surgery is photorefractive keratotomy (PRK) or laser-assisted in situ keratomileusis (LASIK). In some embodiments, the ocular disease, disorder or condition is a disease, disorder, or condition of the corneal stroma. In some embodiments, the disease, disorder, or condition of the corneal stroma is keratoconus, lattice corneal dystrophy, granular corneal dystrophy, macular corneal dystrophy, Schnyder crystalline corneal dystrophy, congenital stromal corneal dystrophy, or fleck corneal dystrophy. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid.

In some aspects, provided herein are methods of treating wound in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating a broken bone in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods of treating a bone lesion in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are methods to rejuvenate or reduce scar formation in a mammal comprising administering to the mammal a pharmaceutical formulation comprising: a modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical composition is free of heparin.

In some aspects, provided herein are pharmaceutical formulations comprising: a modified FGF comprising one or more mutations of human wild type FGF-1 (SEQ ID NO: 109) at positions K9, K10, P11, K12, C16, C83, C117, P134, P136, and V137; and a pharmaceutically acceptable carrier, excipient, or diluent. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical formulation is a liquid formulation. In some embodiments, the pharmaceutical formulation is free of heparin.

In some aspects, provided herein are pharmaceutical formulations comprising: a modified FGF, the modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; and a pharmaceutically acceptable carrier, excipient, or diluent. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical formulation is a liquid formulation. In some embodiments, the pharmaceutical formulation is free of heparin.

In some aspects, provided herein are kits comprising: a modified FGF comprising one or more mutations of human wild type FGF-1 (SEQ ID NO: 109) at positions K9, K10, P11, K12, C16, C83, C117, P134, P136, and V137; a pharmaceutically acceptable carrier, excipient, or diluent; and a container. In some embodiments, the modified FGF comprises an N-terminal truncation comprising one or more of the first 12 residues. In some embodiments, the modified FGF comprises a C-terminal truncation comprising one or more of the last 5 residues. In some embodiments, the modified FGF comprises a N-terminal truncation and a C-terminal truncation, and wherein the N-terminal truncation comprises one or more of the first 12 residues and the C-terminal truncation comprises one or more of the last 5 residues. In some embodiments, the modified FGF is truncated up to the first mutated residue. In some embodiments, the modified FGF is truncated after the last mutated residue. In some embodiments, the modified FGF comprises at least one of the mutations: K12V, C117V, and P134V. In some embodiments, the modified FGF comprises at least one of the mutations: K12I, C117I, and P134I. In some embodiments, the modified FGF further comprises at least one of the mutations: C16A and C83A. In some embodiments, the modified FGF further comprises at least one of the mutations: K9C, K10C, and P11C. In some embodiments, the modified FGF further comprises at least one of the mutations: P136C and V137C. In some embodiments, mutating one or more of residues 9, 10 and 11 to cysteine and mutating one or more of residues 136 and 137 to cysteine introduces a stabilizing disulfide bond between the mutated residues. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical formulation is a liquid formulation. In some embodiments, the pharmaceutical formulation is free of heparin.

In some aspects provided herein are kits comprising: a modified FGF, the modified FGF comprising the sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108; a pharmaceutically acceptable carrier, excipient, or diluent; and a container. In some embodiments, the modified FGF is conjugated to a dressing, a polymer, or a biological matrix. In some embodiments, the biological matrix comprises at least one of sulfhydryl derivatized hydroxyapatite, a polymer or copolymer of polylactide, and a decellularized extracellular matrix. In some embodiments, the modified FGF is conjugated to a nanoparticle. In some embodiments, the modified FGF is conjugated to a sulfhydryl derivatized hydroxyapatite nanoparticle. In some embodiments, the biological matrix is modified to create thiol functional groups capable of forming disulfide bonds with the modified FGF. In some embodiments, a material used to modify the biological matrix comprises at least one of: thiol-modified collagen and thiol-modified hyaluronic acid. In some embodiments, the pharmaceutical formulation is a liquid formulation. In some embodiments, the pharmaceutical formulation is free of heparin.

All publications and patent applications mentioned in this specification are herein incorporated by reference in their entireties to the same extent as if each individual publication or patent application was specifically and individually indicated to be incorporated by reference.

### DETAILED DESCRIPTION

Provided herein are modified fibroblast growth factors (FGFs), pharmaceutical compositions and medicaments, and methods of using such modified FGFs to treat diseases, disorders, or conditions. In some embodiments, the modified FGFs described herein are used to treat corneal disease, disorders and conditions, e.g., corneal endothelial dystrophies such as Fuch's dystrophy (FD). In some embodiments, the modified FGFs described herein are used to treat an ocular disease disorder or condition in a mammal. In some embodiments, the modified FGFs described herein are used to treat a retinal disease in a mammal. In some embodiments, the modified FGFs described herein are used to treat a wound, for example a skin wound, in a mammal. In some embodiments, the modified FGFs described herein are used to treat a broken bone in a mammal. In some embodiments, the modified FGFs described herein are used to treat a bone lesion in a mammal. In some embodiments, the modified FGFs described herein are used to rejuvenate or reduce scar formation in a mammal.

Growth factors are key regulators of the proliferation and migration of the cells of a wide range of mammalian tissues. Fibroblast growth factors (FGFs) comprise a large family of evolutionarily conserved polypeptides involved in a variety of biological processes including morphogenesis, angiogenesis, and tissue remodeling as well as in the pathogenesis of numerous diseases. The various members of this family stimulate the proliferation of a wide spectrum of cells, including those deriving from mesenchymal, endothelial, epithelial and neuroectodermal origin. The biological response of cells to FGF is mediated through specific cell surface receptors (FGFRs).

While administering FGF to patients with disorders, diseases, or conditions (e.g. corneal endothelial dystrophies such as Fuch's dystrophy), seems like a viable treatment, there are several issues that complicate effective realization of FGF-1 as a therapeutic. First, FGF has relatively poor thermal stability in the absence of heparin, which negatively impacts potency and storage lifetimes as well as its potential half-life in vivo. Although inclusion of heparin in the formulation can increase stability, it negatively impacts the binding of FGF-1 to tissue heparans. These are critical issues impacting efficacy and frequency of administration. In addition, heparin is more expensive than FGF-1 to produce, is derived from pigs (with the potential for infectious agents), is naturally heterogeneous in structure and function, and has anti-coagulant activity, which can prevent safely achieving optimal dose levels. Furthermore, some individuals are immunologically sensitized to heparin, which on subsequent exposure can lead to heparin-induced thrombosis (Prechel et al. Seminars in Thrombostasis and Hemostasis. 2012; 38:483-96).

Several members of the FGF family of proteins, including FGF-1, have the potential of providing “angiogenic therapy” for the treatment of ischemic conditions or diseases (i.e., diseases caused by insufficient blood flow to one or more tissues), such as coronary artery disease, peripheral vascular disease, peripheral arterial occlusion or disease (e.g., critical limb ischemia or CLI), etc., by triggering neovascularization of affected tissues. See, e.g., Nikol, S. et al., “Therapeutic Angiogenesis With Intramuscular NV1FGF Improves Amputation-free Survival in Patients With Critical Limb Ischemia,” Mol Ther 16(5):972-978 (2008), the entire contents and disclosure of which are hereby incorporated by reference. In some embodiments, FGF proteins and/or modified FGFs are used for tissue repair and wound healing by triggering angiogenesis and proliferation of fibroblasts involved in healing damaged tissue and filling the wound space with new tissue.

The majority if not all of FGF activity is manifested through interactions with four receptors, including several variants arising from alternate splicing events, that are members of the fibroblast growth factor receptor family. These receptors are variously utilized by the nearly two dozen other FGF subtypes, but FGF-1 is the only one that can bind to every receptor subform. The amino acid sequence of FGF-1 that is commonly expressed (SEQ ID NO: 109) contains 140 amino acid residues commencing with a phenylalanine residue and terminating in an aspartic acid residue. Notably it contains three cysteine residues at positions 16, 83 and 117 that remain in the reduced state even after export from the cell. Three-dimensional analyses have determined that the FGF family has a pseudo threefold symmetry that defines its trefoil structure. As a result of this organization, the N- and C-termini are closely juxtaposed, held in this orientation by an antiparallel β-sheet composed of residues 13-17 and 131-135. The residues extending beyond this portion of the secondary structure extend away from each other.

A number of positions in the sequence of FGF-1 have been substituted with varying effects on the stability and function of the molecule. The three thiol-containing residues of FGF-1 are susceptible to oxidation that results in unfolding and loss of activity. The inventors of the subject matter described herein, have designed modified FGF-1 proteins with substitutions of one or more residues susceptible to oxidation so as to increase protein stability. Further, the inventors of the subject matter described herein have designed modified FGF-1 proteins to maintain the important β1-β12, thus increasing protein stability. The inventors of the subject matter described herein have also discovered other modifications to the FGF-1 protein that confer increased stability.

### Certain Terminology

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “treat,” “treating” or “treatment” include alleviating, abating or ameliorating a disease, disorder or condition symptoms, preventing additional symptoms, ameliorating or preventing the underlying metabolic causes of symptoms, inhibiting the disease, disorder, or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition. The terms “treat,” “treating” or “treatment”, include, but are not limited to, prophylactic and/or therapeutic treatments.

The term “acceptable” or “pharmaceutically acceptable”, with respect to a formulation, composition or ingredient, refers to having no persistent detrimental effect on the general health of the subject being treated or does not abrogate the biological activity or properties of the modified FGF described herein, and is relatively nontoxic.

The term “amelioration” of the symptoms of a particular disease, disorder or condition by administration of a particular modified FGF or pharmaceutical composition refers to any lessening of severity, delay in onset, slowing of progression, or shortening of duration, whether permanent or temporary, lasting or transient that can be attributed to or associated with administration of the modified FGF or pharmaceutical composition.

The term “combination” or “pharmaceutical combination” as used herein, means a product that results from the mixing or combining of more than one active ingredient and includes both fixed and non-fixed combinations of the active ingredients. The term “fixed combination” means that one active ingredient (e.g. a modified FGF) and a co-agent are both administered to a patient simultaneously in the form of a single entity or dosage. The term “non-fixed combination” means that one active ingredient (e.g. a modified FGF) and a co-agent are administered to a patient as separate entities either simultaneously, concurrently or sequentially with no specific intervening time limits, wherein such administration provides effective levels of the two agents in the body of the patient. The latter also applies to cocktail therapy, e.g. the administration of three or more active ingredients.

The term “pharmaceutical composition” as used herein refers to one or more modified FGFs with one or more other chemical components, such as carriers, stabilizers, diluents, dispersing agents, suspending agents, thickening agents, and/or excipients. The pharmaceutical composition facilitates administration of the modified FGF to an organism. Multiple techniques of administering a modified FGF exist in the art including, but not limited to: topical, ophthalmic, intraocular, periocular, intravenous, oral, aerosol, parenteral, and administration.

The term “carrier,” as used herein, refers to relatively nontoxic chemical compounds or agents that facilitate the incorporation of an agent of interest (e.g., a modified FGF) into cells or tissues.

The term “diluent” refers to chemical compounds that are used to dilute the agent of interest (e.g., a modified FGF) prior to delivery. Diluents can also be used to stabilize agents because they can provide a more stable environment. Salts dissolved in buffered solutions (which also can provide pH control or maintenance) are utilized as diluents in the art, including, but not limited to a phosphate buffered saline solution.

The terms “co-administration” or the like, are meant to encompass administration of the selected agents (e.g., a modified FGF or composition thereof and a co-agent) to a single patient, and are intended to include treatment regimens in which the agents are administered by the same or different route of administration or at the same or different time.

The terms “effective amount” or “therapeutically effective amount,” refer to a sufficient amount of a modified FGF, agent, combination or pharmaceutical composition described herein administered which will relieve to some extent one or more of the symptoms of the disease, disorder or condition being treated. The result can be reduction and/or alleviation of the signs, symptoms, or causes of a disease, or any other desired alteration of a biological system. For example, an “effective amount” for therapeutic uses is the amount of the modified FGF, agent, combination or pharmaceutical composition required to provide a desired pharmacologic effect, therapeutic improvement, or clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. It is understood that “an effect amount” can vary from subject to subject due to variation in metabolism of the modified FGF, combination, or pharmaceutical composition, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “prophylactically effective amount,” refers that amount of a modified FGF, compound, agent, combination or pharmaceutical composition described herein applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” or “patient” as used herein, refers to an animal, which is the object of treatment, observation or experiment. By way of example only, a subject may be, but is not limited to, a mammal including, but not limited to, a human.

The terms “enhance” or “enhancing” means to increase or prolong either in potency or duration a desired effect. By way of example, “enhancing” the effect of therapeutic agents singly or in combination refers to the ability to increase or prolong, either in potency, duration and/or magnitude, the effect of the agents on the treatment of a disease, disorder or condition. When used in a patient, amounts effective for this use will depend on the severity and course of the disease, disorder or condition, previous therapy, the patient's health status and response to the drugs, and the judgment of the treating physician.

The term “modulate,” means to interact with a target (e.g., a FGF receptor) either directly or indirectly so as to alter the activity of the target, including, by way of example only, to enhance the activity of the target, to inhibit or antagonize the activity of the target, to limit the activity of the target, or to extend the activity of the target. In some embodiments, modified FGFs and pharmaceutical compositions described herein can modulate the activity of one or more respective targets (e.g., one or more FGF receptors). In some embodiments, the modified FGFs described herein modulate (e.g., increase) the activity of one or more FGF receptors on a cell (e.g., a corneal endothelial cell), resulting, e.g., in cell migration and/or cell proliferation.

As used herein, the term “target” refers to a biological molecule (e.g., a target protein or protein complex), such as an FGF receptor, or a portion of a biological molecule capable of being bound by a selective binding agent (e.g., a modified FGF) or pharmaceutical composition described herein. As used herein, the term “non-target” refers to a biological molecule or a portion of a biological molecule that is not selectively bound by a selective binding agent or pharmaceutical composition described herein.

The term “target activity” or “cell response” refers to a biological activity capable of being modulated by a modified FGF or any cellular response that results from the binding of a modified FGF to a FGF receptor. Certain exemplary target activities and cell responses include, but are not limited to, binding affinity, signal transduction, gene expression, cell migration, cell proliferation, cell differentiation, and amelioration of one or more symptoms associated with an ocular disease, disorder or condition.

The term “amino acid” refers to the molecules composed of terminal amine and carboxylic acid functional groups with a carbon atom between the terminal amine and carboxylic acid functional groups sometimes containing a side chain functional group attached to the carbon atom (e.g. a methoxy functional group, which forms the amino acid serine). Typically, amino acids are classified as natural and non-natural. Examples of natural amino acids include glycine, alanine, valine, leucine, isoleucine, proline, phenylananine, tyrosine, tryptophan, serine, threonine, cysteine, methionine, asparagine, glutamine, lysine, arginine, histidine, aspartate, and glutamate, among others. Examples of non-natural amino acids include L-3,4-dihydroxyphenylalanine, 2-aminobutyric acid, dehydralanine, g-carboxyglutamic acid, carnitine, gamma-aminobutyric acid, hydroxyproline, and selenomethionine, among others. In the context of this specification it should be appreciated that the amino acids may be the L-optical isomer or the D-optical isomer.

### Modified Fibroblast Growth Factors (FGFs)

Embodiments disclosed herein relate to a modified FGF or a pharmaceutical composition comprising a modified FGF. A modified FGF, as used herein, refers to a wild-type or native FGF that includes a substitution or mutation of one or more different amino acid residues and/or one or more deletions of one or more amino acid residues and/or one or more additions of one or more amino acid residues. The wild-type or native FGF that includes the modification(s) can be any member of the FGF family, including FGF-1 (SEQ ID NO: 109), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23, and FGF-24. FGFs stimulate a family seven FGF receptor isoforms, and each FGF stimulates a different pattern of receptors to achieve its specific effect (Ornitz et al. The Journal of biological chemistry. 1996; 271(25):15292-7; Zhang et al. The Journal of biological chemistry. 2006; 281(23):15694-700).

In some embodiments, the modified FGF is a modified FGF or a modified FGF-2. In some embodiments, the modified FGF is a modified FGF. In some embodiments, modified FGF is preferable since it binds to and stimulates all seven FGF receptor isoforms (Ornitz et al. The Journal of biological chemistry. 1996; 271(25):15292-7).

In some embodiments, the modified FGF is thermostable. As used herein, a thermostable FGF (e.g., a thermostable FGF-1) refers to an FGF having a modified (e.g. mutated truncated, extended) amino acid sequence relative to the wild type FGF sequence that is also more stable than the wild type FGF under the same conditions. Examples of mutations capable of conferring thermostability to FGF (e.g., FGF-1) and methods for assessing thermostability are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404, 2013/0130983; and in Xia et al. PloS one. 2012; 7(11):e48210. In some embodiments, positions 9, 10, 11, 12, 1, 83, 117, 134, 136, and/or 137 are mutated in FGF-1 to generate a modified FGF that is more thermostable compared to FGF-1 (i.e. SEQ ID NO: 109). In various embodiments reducing the number of native FGF-1 thiols increases thermostability. In various embodiments, one or more thiols are introduced to maintain the β1-β12 interaction so as to increase thermostability.

In some embodiments, the modified FGF includes one or more modifications that reduce the number of reactive thiols (e.g., free cysteines). Examples such modifications in FGF (e.g., FGF-1) are described, for example, in U.S. Pat. Nos. 5,223,483; 5,312,911; 5,409,897; 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. 2012; 7(11):e48210. In some embodiments, positions 16, 83 and/or 117 are mutated in FGF-1 to generate a modified FGF that has a reduced number of reactive thiols. In various embodiments reducing the number of native FGF-1 thiols increases stability.

The modified FGFs described herein are uniquely suited for application in the eye. Because modified FGFs described herein can be administered without exogenous heparin in the formulation for stability, they can be formulated and applied without heparin and thus are more able to bind to the tissue heparans. Such modified FGFs have a high affinity for tissue heparans that are exposed in a surgical, traumatic or dystrophic conditions and disease-states and so bind to diseased tissue on application. In addition, the modified FGFs being more thermally stable are suitable for formulation and storage at room temperature. The stability of the modified FGFs also makes them suitable for administration in both solution (e.g., immediate release) and sustained-release formulations.

The modified FGFs described herein are also uniquely suited for application to regeneration of tissue without conversion of the proliferating cells to fibroblasts or the induction of a fibroblastic or myofibroblastic phenotype (epi- or endo-thelilal mesenchymal transition or EMT) or scar formation. FGF-1 is known to inhibit EMT (see for example Ramos et al., Am J Physiol Lung Cell Mol Physiol 2010; 299:L222-L231). Because modified FGFs are stabilized and have longer half-lives, they can provide powerful and consistent suppression of EMT. In addition, certain FGFs are particularly potent in suppressing the fibroblastic transition.

The modified FGFs described herein are also uniquely suited to treat a wound, for example a skin wound. In some embodiments, modified FGFs described herein are administered as a dressing to a wound site by bringing immobilized FGF directly into contact with the wound and releasing the modified FGF into the wound. In some embodiments, the modified FGF is released into the wound over time at one or more doses. In some embodiments, releasing the modified FGF to the wound site using a dressing increases the efficacy of the treatment and decreases the potential for side effects distal to the wound site.

Modified FGFs for use in the compositions and methods described herein can be any modified FGF known in the art or described herein that is thermostable, comprises a reduced number of reactive thiols, and/or remains biologically active when administered without heparin as measured by any suitable assay known in the art or described herein.

In some embodiments, the modified FGF is any one of the modified FGF proteins disclosed in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. 2012; 7(11):e48210. In some embodiments, the modified FGF is a wild-type FGF-1 (e.g. SEQ ID NO: 109) that has been modified at one or more of the positions 9, 10, 11, 12, 16, 83, 117, 134, 136, and 137 with, e.g., Val, Ile Thr, Cys, Ala. In various embodiments, modification of one or more of the residues 9, 10, 11, 12, 16, 83, 117, 134, 136, and 137 confer increased stability of the modified FGF.

In some embodiments, the modified FGF comprises the sequence of SEQ ID NO: 109, wherein two or more FGF-1 amino acids are substituted with cysteine so as to introduce one or more disulfide bonds between modified amino acids and wild-type amino acids, or create disulfide bonds between two modified amino acids. In various embodiments, one or more N-terminal residues and one or more C-terminal residues are substituted with cysteine so as to introduce a disulfide bond between two modified amino acids. In various embodiments, introducing one or more disulfide bonds confer increased stability of the modified FGF. In some embodiments, one or more of residues 9, 10, and 11 are substituted to cysteine and one or more of residues 136 and 137 are substituted to cysteine. In various embodiments, substituting one or more of residues 9, 10, and 11 and one or more of 136 and 137 introduces a disulfide in the modified FGF. As a non-limiting example residue 9 and residue 136 are substituted to cysteine so as to introduce a disulfide bond. As a non-limiting example residue 9 and residue 137 are substituted to cysteine so as to introduce a disulfide bond. As a non-limiting example residue 10 and residue 136 are substituted to cysteine so as to introduce a disulfide bond. As a non-limiting example residue 10 and residue 137 are substituted to cysteine so as to introduce a disulfide bond. As a non-limiting example residue 11 and residue 136 are substituted to cysteine so as to introduce a disulfide bond. As a non-limiting example residue 11 and residue 137 are substituted to cysteine so as to introduce a disulfide bond.

In some embodiments, the modified FGF is a truncated FGF-1. In various embodiments, the truncated FGF-1 comprises the sequence of SEQ ID NO: 109, wherein one or more N-terminal residues are deleted. In various embodiments, one or more of the first 15 residues are deleted. In various embodiments, one or more of the first 12 residues are deleted. In various embodiments, one or more of the first 5 residues are deleted. In various embodiments, the first 5 residues of SEQ ID NO: 109 are deleted. In various embodiments, one or more C-terminal residues are deleted to generate the truncated FGF-1. In various embodiments, one or more of the last 15 residues are deleted. In various embodiments, one or more of the last 5 residues are deleted. In various embodiments, one or more of the last 3 residues are deleted. In various embodiments, the last 3 residues are deleted. In various embodiments, one or more N-terminal residues and one or more C-terminal residues are deleted to generate the truncated FGF-1. In various embodiments, one or more of the first 15 residues and last 15 residues of FGF-1 are deleted. In various embodiments, one or more of the first 12 residues and last 5 residues of FGF-1 are deleted. In various embodiments, one or more of the first 5 and last 3 residues of FGF-1 are deleted. In various embodiments, the first 5 residues and last 3 residues of FGF-1 are deleted.

In some embodiments, the modified FGF is generated using FGF-1 as a template. In some embodiments, substitutions K12V, C117V and P134V are incorporated so as to increase thermostability. In some embodiments, isoleucines are used instead of valine to give the substitutions K12I, C117I and P134I. In various embodiments, the increased hydrophobicity of Ile provides increased van der Waals interactions and provides greater stability compared to the modified FGf-1 generated using the K12V, C117V and P134V substitutions. In some embodiments, the remaining two naturally occurring cysteine residues at 16 and 87 (in the 140 numbering) are substituted with alanine residues. In various embodiments, substituting all naturally occurring cysteine residues eliminates a possibility of oxidation damage. In some embodiments cysteine residues will be substituted in place of residues 9, 10 or 11 and at residues 136 or 137 (note the positions are based on the in the 140 numbering). The position of the substitutions at residues 9, 10 or 11 and at residues 136 or 137 put them outside the limits of the canonical B-sheet backbone H-bonding pattern where there should be enough additional flexibility to allow a disulfide bond to form between the cysteine pairs. In some embodiments, one or more N-terminal residues are deleted and one or more C-terminal residues are deleted, thus, the modified FGF incorporates one or more of the aforementioned substitutions and is truncated at the N-terminal and/or C terminal end.

In some embodiments, the modified FGF is FGF-1 that is truncated on both terminal ends is used as a template. In various embodiments, the terminal truncations eliminate residues 1-5 and 138-140 resulting in a modified FGF with 132 residues. In some embodiments, substitutions K12V, C117V and P134V are incorporated so as to increase thermostability. In some embodiments, isoleucine are used instead of valine to give the substitutions K12I, C117I and P134I. In various embodiments, the increased hydrophobicity of Ile provides increased van der Waals interactions and provides greater stability compared to the modified FGf-1 generated using the K12V, C117V and P134V substitutions. In some embodiments, the remaining two naturally occurring cysteine residues at 16 and 87 (in the 140 numbering) are substituted with alanine residues. In various embodiments, substituting all naturally occurring cysteine residues eliminates a possibility of oxidation damage. In some embodiments cysteine residues will be substituted in place of residues 9, 10 or 11 and at residues 136 or 137 (note the positions are based on the in the 140 numbering). The position of the substitutions at residues 9, 10 or 11 and at residues 136 or 137 put them outside the limits of the canonical B-sheet backbone H-bonding pattern where there should be enough additional flexibility to allow a disulfide bond to form between the cysteine pairs.

In some embodiments, the modified FGF is human wild-type FGF protein (SEQ ID NO: 109 modified at position 12 with, e.g., Val, Ile, Ser, Thr, Cys, Ala or another amino acid. In some embodiments, the modified FGF is human wild-type FGF protein with the mutation Lys12Val. In some embodiments, the modified FGF is human wild-type FGF protein with the mutation Lys12Ile.

In some embodiments, the modified FGF is human wild-type FGF protein (SEQ ID NO: 109 modified at position 16 with, e.g., Val, Ile, Ser, Thr, Cys, Ala or another amino acid. In some embodiments, the modified FGF is human wild-type FGF protein with the mutation Cys16Ala.

In some embodiments, the modified FGF is human wild-type FGF protein (SEQ ID NO: 109 modified at position 83 with, e.g., Val, Ile, Ser, Thr, Cys, Ala or another amino acid. In some embodiments, the modified FGF is human wild-type FGF protein with the mutation Cys83Ala.

In some embodiments, the modified FGF is human wild-type FGF protein (SEQ ID NO: 109 modified at position 117 with, e.g., Val, Ile, Ser, Thr, Cys, Ala or another amino acid. In some embodiments, the modified FGF is human wild-type FGF protein with the mutation Cys117Val. In some embodiments, the modified FGF is human wild-type FGF protein with the mutation Cys117Ile.

In some embodiments, the modified FGF is human wild-type FGF protein (SEQ ID NO: 109 modified at position 134 with, e.g., Val, Ile, Ser, Thr, Cys, Ala or another amino acid. In some embodiments, the modified FGF is human wild-type FGF protein with the mutation Pro134Val. In some embodiments, the modified FGF is human wild-type FGF protein with the mutation Pro134Ile.

In some embodiments, the modified FGF is human wild-type FGF protein (SEQ ID NO: 109 modified at position 9 with, e.g., Val, Ile, Ser, Thr, Cys, Ala or another amino acid. In some embodiments, the modified FGF is human wild-type FGF protein with the mutation Lys9Cys.

In some embodiments, the modified FGF is human wild-type FGF protein (SEQ ID NO: 109 modified at position 10 with, e.g., Val, Ile, Ser, Thr, Cys, Ala or another amino acid. In some embodiments, the modified FGF is human wild-type FGF protein with the mutation Lys10Cys.

In some embodiments, the modified FGF is human wild-type FGF protein (SEQ ID NO: 109 modified at position 11 with, e.g., Val, Ile, Ser, Thr, Cys, Ala or another amino acid. In some embodiments, the modified FGF is human wild-type FGF protein with the mutation Lys10Cys.

In some embodiments, the modified FGF is human wild-type FGF protein (SEQ ID NO: 109 modified at position 136 with, e.g., Val, Ile, Ser, Thr, Cys, Ala or another amino acid. In some embodiments, the modified FGF is human wild-type FGF protein with the mutation Pro136Cys.

In some embodiments, the modified FGF is human wild-type FGF protein (SEQ ID NO: 109 modified at position 137 with, e.g., Val, Ile, Ser, Thr, Cys, Ala or another amino acid. In some embodiments, the modified FGF is human wild-type FGF protein with the mutation Val137Cys.

In some embodiments, the modified FGF is a wild-type FGF-1 (e.g. SEQ ID NO: 109) that has been modified at position 12 with, e.g., Val, Ile, Ser, Thr, Cys, Ala or another amino acid. In some embodiments, the modified FGF that has been modified at position 12 is further modified at position 16 with, e.g., Val, Ile, Ser, Thr, Cys, Ala or another amino acid. In some embodiments, the modified FGF that has been modified at position 12 is further modified at position 83 with, e.g., Val, Ile, Ser, Thr, Cys, Ala or another amino acid. In some embodiments, the modified FGF that has been modified at position 12 is further modified at position 117 with, e.g., Val, Ile, Ser, Thr, Cys, Ala or another amino acid. In some embodiments, the modified FGF that has been modified at position 12 is further modified at position 134 with, e.g., Val, Ile, Ser, Thr, Cys, Ala or another amino acid. In some embodiments, the modified FGF that has been modified at position 12 is further modified at position 9 with, e.g., Val, Ile, Ser, Thr, Cys, Ala or another amino acid. In some embodiments, the modified FGF that has been modified at position 12 is further modified at position 10 with, e.g., Val, Ile, Ser, Thr, Cys, Ala or another amino acid. In some embodiments, the modified FGF that has been modified at position 12 is further modified at position 11 with, e.g., Val, Ile, Ser, Thr, Cys, Ala or another amino acid. In some embodiments, the modified FGF that has been modified at position 12 is further modified at position 136 with, e.g., Val, Ile, Ser, Thr, Cys, Ala or another amino acid. In some embodiments, the modified FGF that has been modified at position 12 is further modified at position 137 with, e.g., Val, Ile, Ser, Thr, Cys, Ala or another amino acid. In some embodiments, the modified FGF that has been modified at position 12 is further modified at position 9 and position 136 with, e.g., Val, Ile, Ser, Thr, Cys, Ala or another amino acid. In some embodiments, the modified FGF that has been modified at position 12 is further modified at position 9 and position 137 with, e.g., Val, Ile, Ser, Thr, Cys, Ala or another amino acid. In some embodiments, the modified FGF that has been modified at position 12 is further modified at position 10 and position 136 with, e.g., Val, Ile, Ser, Thr, Cys, Ala or another amino acid. In some embodiments, the modified FGF that has been modified at position 12 is further modified at position 10 and position 137 with, e.g., Val, Ile, Ser, Thr, Cys, Ala or another amino acid. In some embodiments, the modified FGF that has been modified at position 12 is further modified at position 11 and position 136 with, e.g., Val, Ile, Ser, Thr, Cys, Ala or another amino acid. In some embodiments, the modified FGF that has been modified at position 12 is further modified at position 11 and position 137 with, e.g., Val, Ile, Ser, Thr, Cys, Ala or another amino acid.

In some embodiments, the modified FGF that has been modified at position 12 and position 16 is further modified at position 83 with, e.g., Val, Ile, Ser, Thr, Cys, Ala or another amino acid. In some embodiments, the modified FGF that has been modified at position 12 and position 16 is further modified at position 117 with, e.g., Val, Ile, Ser, Thr, Cys, Ala or another amino acid. In some embodiments, the modified FGF that has been modified at position 12 and position 16 is further modified at position 134 with, e.g., Val, Ile, Ser, Thr, Cys, Ala or another amino acid. In some embodiments, the modified FGF that has been modified at position 12 and position 16 is further modified at position 9 with, e.g., Val, Ile, Ser, Thr, Cys, Ala or another amino acid. In some embodiments, the modified FGF that has been modified at position 12 and position 16 is further modified at position 10 with, e.g., Val, Ile, Ser, Thr, Cys, Ala or another amino acid. In some embodiments, the modified FGF that has been modified at position 12 and position 16 is further modified at position 11 with, e.g., Val, Ile, Ser, Thr, Cys, Ala or another amino acid. In some embodiments, the modified FGF that has been modified at position 12 and position 16 is further modified at position 136 with, e.g., Val, Ile, Ser, Thr, Cys, Ala or another amino acid. In some embodiments, the modified FGF that has been modified at position 12 and position 16 is further modified at position 137 with, e.g., Val, Ile, Ser, Thr, Cys, Ala or another amino acid. In some embodiments, the modified FGF that has been modified at position 12 and position 16 is further modified at position 9 and position 136 with, e.g., Val, Ile, Ser, Thr, Cys, Ala or another amino acid. In some embodiments, the modified FGF that has been modified at position 12 and position 16 is further modified at position 9 and position 137 with, e.g., Val, Ile, Ser, Thr, Cys, Ala or another amino acid. In some embodiments, the modified FGF that has been modified at position 12 and position 16 is further modified at position 10 and position 136 with, e.g., Val, Ile, Ser, Thr, Cys, Ala or another amino acid. In some embodiments, the modified FGF that has been modified at position 12 and position 16 is further modified at position 10 and position 137 with, e.g., Val, Ile, Ser, Thr, Cys, Ala or another amino acid. In some embodiments, the modified FGF that has been modified at position 12 and position 16 is further modified at position 11 and position 136 with, e.g., Val, Ile, Ser, Thr, Cys, Ala or another amino acid. In some embodiments, the modified FGF that has been modified at position 12 and position 16 is further modified at position 11 and position 137 with, e.g., Val, Ile, Ser, Thr, Cys, Ala or another amino acid.

In some embodiments, the modified FGF that has been modified at position 12, 16 and 83 is further modified at position 117 with, e.g., Val, Ile, Ser, Thr, Cys, Ala or another amino acid. In some embodiments, the modified FGF that has been modified at position 12, 16 and 83 is further modified at position 134 with, e.g., Val, Ile, Ser, Thr, Cys, Ala or another amino acid. In some embodiments, the modified FGF that has been modified at position 12, 16, and 83 is further modified at position 9 with, e.g., Val, Ile, Ser, Thr, Cys, Ala or another amino acid. In some embodiments, the modified FGF that has been modified at position 12, 16, and 83 is further modified at position 10 with, e.g., Val, Ile, Ser, Thr, Cys, Ala or another amino acid. In some embodiments, the modified FGF that has been modified at position 12, 16, and 83 is further modified at position 11 with, e.g., Val, Ile, Ser, Thr, Cys, Ala or another amino acid. In some embodiments, the modified FGF that has been modified at position 12, 16, and 83 is further modified at position 136 with, e.g., Val, Ile, Ser, Thr, Cys, Ala or another amino acid. In some embodiments, the modified FGF that has been modified at position 12, 16, and 83 is further modified at position 137 with, e.g., Val, Ile, Ser, Thr, Cys, Ala or another amino acid. In some embodiments, the modified FGF that has been modified at position 12, 16, and 83 is further modified at position 9 and position 136 with, e.g., Val, Ile, Ser, Thr, Cys, Ala or another amino acid. In some embodiments, the modified FGF that has been modified at position 12, 16, and 83 is further modified at position 9 and position 137 with, e.g., Val, Ile, Ser, Thr, Cys, Ala or another amino acid. In some embodiments, the modified FGF that has been modified at position 12, 16, and 83 is further modified at position 10 and position 136 with, e.g., Val, Ile, Ser, Thr, Cys, Ala or another amino acid. In some embodiments, the modified FGF that has been modified at position 12, 16, and 83 is further modified at position 10 and position 137 with, e.g., Val, Ile, Ser, Thr, Cys, Ala or another amino acid. In some embodiments, the modified FGF that has been modified at position 12, 16, and 83 is further modified at position 11 and position 136 with, e.g., Val, Ile, Ser, Thr, Cys, Ala or another amino acid. In some embodiments, the modified FGF that has been modified at position 12, 16, and 83 is further modified at position 11 and position 137 with, e.g., Val, Ile, Ser, Thr, Cys, Ala or another amino acid.

In some embodiments, the modified FGF that has been modified at position 12, 16, 83 and 117 is further modified at position 134 with, e.g., Val, Ile, Ser, Thr, Cys, Ala or another amino acid. In some embodiments, the modified FGF that has been modified at position 12, 16, 83, and 117 is further modified at position 9 with, e.g., Val, Ile, Ser, Thr, Cys, Ala or another amino acid. In some embodiments, the modified FGF that has been modified at position 12, 16, 83, and 117 is further modified at position 10 with, e.g., Val, Ile, Ser, Thr, Cys, Ala or another amino acid. In some embodiments, the modified FGF that has been modified at position 12, 16, 83, and 117 is further modified at position 11 with, e.g., Val, Ile, Ser, Thr, Cys, Ala or another amino acid. In some embodiments, the modified FGF that has been modified at position 12, 16, 83, and 117 is further modified at position 136 with, e.g., Val, Ile, Ser, Thr, Cys, Ala or another amino acid. In some embodiments, the modified FGF that has been modified at position 12, 16, 83, and 117 is further modified at position 137 with, e.g., Val, Ile, Ser, Thr, Cys, Ala or another amino acid. In some embodiments, the modified FGF that has been modified at position 12, 16, 83, and 117 is further modified at position 9 and position 136 with, e.g., Val, Ile, Ser, Thr, Cys, Ala or another amino acid. In some embodiments, the modified FGF that has been modified at position 12, 16, 83, and 117 is further modified at position 9 and position 137 with, e.g., Val, Ile, Ser, Thr, Cys, Ala or another amino acid. In some embodiments, the modified FGF that has been modified at position 12, 16, 83, and 117 is further modified at position 10 and position 136 with, e.g., Val, Ile, Ser, Thr, Cys, Ala or another amino acid. In some embodiments, the modified FGF that has been modified at position 12, 16, 83, and 117 is further modified at position 10 and position 137 with, e.g., Val, Ile, Ser, Thr, Cys, Ala or another amino acid. In some embodiments, the modified FGF that has been modified at position 12, 16, 83, and 117 is further modified at position 11 and position 136 with, e.g., Val, Ile, Ser, Thr, Cys, Ala or another amino acid. In some embodiments, the modified FGF that has been modified at position 12, 16, 83, and 117 is further modified at position 11 and position 137 with, e.g., Val, Ile, Ser, Thr, Cys, Ala or another amino acid.

In some embodiments, the modified FGF that has been modified at position 12 and position 83 is further modified at position 117 with, e.g., Val, Ile, Ser, Thr, Cys, Ala or another amino acid. In some embodiments, the modified FGF that has been modified at position 12 and position 83 is further modified at position 134 with, e.g., Val, Ile, Ser, Thr, Cys, Ala or another amino acid. In some embodiments, the modified FGF that has been modified at position 12 and position 83 is further modified at position 9 with, e.g., Val, Ile, Ser, Thr, Cys, Ala or another amino acid. In some embodiments, the modified FGF that has been modified at position 12 and position 83 is further modified at position 10 with, e.g., Val, Ile, Ser, Thr, Cys, Ala or another amino acid. In some embodiments, the modified FGF that has been modified at position 12 and position 83 is further modified at position 11 with, e.g., Val, Ile, Ser, Thr, Cys, Ala or another amino acid. In some embodiments, the modified FGF that has been modified at position 12 and position 83 is further modified at position 136 with, e.g., Val, Ile, Ser, Thr, Cys, Ala or another amino acid. In some embodiments, the modified FGF that has been modified at position 12 and position 83 is further modified at position 137 with, e.g., Val, Ile, Ser, Thr, Cys, Ala or another amino acid. In some embodiments, the modified FGF that has been modified at position 12 and position 83 is further modified at position 9 and position 136 with, e.g., Val, Ile, Ser, Thr, Cys, Ala or another amino acid. In some embodiments, the modified FGF that has been modified at position 12 and position 83 is further modified at position 9 and position 137 with, e.g., Val, Ile, Ser, Thr, Cys, Ala or another amino acid. In some embodiments, the modified FGF that has been modified at position 12 and position 83 is further modified at position 10 and position 136 with, e.g., Val, Ile, Ser, Thr, Cys, Ala or another amino acid. In some embodiments, the modified FGF that has been modified at position 12 and position 83 is further modified at position 10 and position 137 with, e.g., Val, Ile, Ser, Thr, Cys, Ala or another amino acid. In some embodiments, the modified FGF that has been modified at position 12 and position 83 is further modified at position 11 and position 136 with, e.g., Val, Ile, Ser, Thr, Cys, Ala or another amino acid. In some embodiments, the modified FGF that has been modified at position 12 and position 83 is further modified at position 11 and position 137 with, e.g., Val, Ile, Ser, Thr, Cys, Ala or another amino acid.

In some embodiments, the modified FGF that has been modified at position 12 and position 117 is further modified at position 134 with, e.g., Val, Ile, Ser, Thr, Cys, Ala or another amino acid. In some embodiments, the modified FGF that has been modified at position 12 and position 117 is further modified at position 9 with, e.g., Val, Ile, Ser, Thr, Cys, Ala or another amino acid. In some embodiments, the modified FGF that has been modified at position 12 and position 117 is further modified at position 10 with, e.g., Val, Ile, Ser, Thr, Cys, Ala or another amino acid. In some embodiments, the modified FGF that has been modified at position 12 and position 117 is further modified at position 11 with, e.g., Val, Ile, Ser, Thr, Cys, Ala or another amino acid. In some embodiments, the modified FGF that has been modified at position 12 and position 117 is further modified at position 136 with, e.g., Val, Ile, Ser, Thr, Cys, Ala or another amino acid. In some embodiments, the modified FGF that has been modified at position 12 and position 117 is further modified at position 137 with, e.g., Val, Ile, Ser, Thr, Cys, Ala or another amino acid. In some embodiments, the modified FGF that has been modified at position 12 and position 117 is further modified at position 9 and position 136 with, e.g., Val, Ile, Ser, Thr, Cys, Ala or another amino acid. In some embodiments, the modified FGF that has been modified at position 12 and position 117 is further modified at position 9 and position 137 with, e.g., Val, Ile, Ser, Thr, Cys, Ala or another amino acid. In some embodiments, the modified FGF that has been modified at position 12 and position 117 is further modified at position 10 and position 136 with, e.g., Val, Ile, Ser, Thr, Cys, Ala or another amino acid. In some embodiments, the modified FGF that has been modified at position 12 and position 117 is further modified at position 10 and position 137 with, e.g., Val, Ile, Ser, Thr, Cys, Ala or another amino acid. In some embodiments, the modified FGF that has been modified at position 12 and position 117 is further modified at position 11 and position 136 with, e.g., Val, Ile, Ser, Thr, Cys, Ala or another amino acid. In some embodiments, the modified FGF that has been modified at position 12 and position 117 is further modified at position 11 and position 137 with, e.g., Val, Ile, Ser, Thr, Cys, Ala or another amino acid.

In some embodiments, the modified FGF that has been modified at position 12 and position 134 is further modified at position 134 with, e.g., Val, Ile, Ser, Thr, Cys, Ala or another amino acid. In some embodiments, the modified FGF that has been modified at position 12 and position 134 is further modified at position 9 with, e.g., Val, Ile, Ser, Thr, Cys, Ala or another amino acid. In some embodiments, the modified FGF that has been modified at position 12 and position 134 is further modified at position 10 with, e.g., Val, Ile, Ser, Thr, Cys, Ala or another amino acid. In some embodiments, the modified FGF that has been modified at position 12 and position 134 is further modified at position 11 with, e.g., Val, Ile, Ser, Thr, Cys, Ala or another amino acid. In some embodiments, the modified FGF that has been modified at position 12 and position 134 is further modified at position 136 with, e.g., Val, Ile, Ser, Thr, Cys, Ala or another amino acid. In some embodiments, the modified FGF that has been modified at position 12 and position 134 is further modified at position 137 with, e.g., Val, Ile, Ser, Thr, Cys, Ala or another amino acid. In some embodiments, the modified FGF that has been modified at position 12 and position 134 is further modified at position 9 and position 136 with, e.g., Val, Ile, Ser, Thr, Cys, Ala or another amino acid. In some embodiments, the modified FGF that has been modified at position 12 and position 134 is further modified at position 9 and position 137 with, e.g., Val, Ile, Ser, Thr, Cys, Ala or another amino acid. In some embodiments, the modified FGF that has been modified at position 12 and position 134 is further modified at position 10 and position 136 with, e.g., Val, Ile, Ser, Thr, Cys, Ala or another amino acid. In some embodiments, the modified FGF that has been modified at position 12 and position 134 is further modified at position 10 and position 137 with, e.g., Val, Ile, Ser, Thr, Cys, Ala or another amino acid. In some embodiments, the modified FGF that has been modified at position 12 and position 134 is further modified at position 11 and position 136 with, e.g., Val, Ile, Ser, Thr, Cys, Ala or another amino acid. In some embodiments, the modified FGF that has been modified at position 12 and position 134 is further modified at position 11 and position 137 with, e.g., Val, Ile, Ser, Thr, Cys, Ala or another amino acid.

In some embodiments, the modified FGF that has been modified at position 12, 83, and 117 is further modified at position 134 with, e.g., Val, Ile, Ser, Thr, Cys, Ala or another amino acid. In some embodiments, the modified FGF that has been modified at position 12, 83, and 117 is further modified at position 9 with, e.g., Val, Ile, Ser, Thr, Cys, Ala or another amino acid. In some embodiments, the modified FGF that has been modified at position 12, 83, and 117 is further modified at position 10 with, e.g., Val, Ile, Ser, Thr, Cys, Ala or another amino acid. In some embodiments, the modified FGF that has been modified at position 12, 83, and 117 is further modified at position 11 with, e.g., Val, Ile, Ser, Thr, Cys, Ala or another amino acid. In some embodiments, the modified FGF that has been modified at position 12, 83, and 117 is further modified at position 136 with, e.g., Val, Ile, Ser, Thr, Cys, Ala or another amino acid. In some embodiments, the modified FGF that has been modified at position 12, 83, and 117 is further modified at position 137 with, e.g., Val, Ile, Ser, Thr, Cys, Ala or another amino acid. In some embodiments, the modified FGF that has been modified at position 12, 83, and 117 is further modified at position 9 and position 136 with, e.g., Val, Ile, Ser, Thr, Cys, Ala or another amino acid. In some embodiments, the modified FGF that has been modified at position 12, 83, and 117 is further modified at position 9 and position 137 with, e.g., Val, Ile, Ser, Thr, Cys, Ala or another amino acid. In some embodiments, the modified FGF that has been modified at position 12, 83, and 117 is further modified at position 10 and position 136 with, e.g., Val, Ile, Ser, Thr, Cys, Ala or another amino acid. In some embodiments, the modified FGF that has been modified at position 12, 83, and 117 is further modified at position 10 and position 137 with, e.g., Val, Ile, Ser, Thr, Cys, Ala or another amino acid. In some embodiments, the modified FGF that has been modified at position 12, 83, and 117 is further modified at position 11 and position 136 with, e.g., Val, Ile, Ser, Thr, Cys, Ala or another amino acid. In some embodiments, the modified FGF that has been modified at position 12, 83, and 117 is further modified at position 11 and position 137 with, e.g., Val, Ile, Ser, Thr, Cys, Ala or another amino acid.

In some embodiments, the modified FGF that has been modified at position 12, 83, and 134 is further modified at position 134 with, e.g., Val, Ile, Ser, Thr, Cys, Ala or another amino acid. In some embodiments, the modified FGF that has been modified at position 12, 83, and 134 is further modified at position 9 with, e.g., Val, Ile, Ser, Thr, Cys, Ala or another amino acid. In some embodiments, the modified FGF that has been modified at position 12, 83, and 134 is further modified at position 10 with, e.g., Val, Ile, Ser, Thr, Cys, Ala or another amino acid. In some embodiments, the modified FGF that has been modified at position 12, 83, and 134 is further modified at position 11 with, e.g., Val, Ile, Ser, Thr, Cys, Ala or another amino acid. In some embodiments, the modified FGF that has been modified at position 12, 83, and 134 is further modified at position 136 with, e.g., Val, Ile, Ser, Thr, Cys, Ala or another amino acid. In some embodiments, the modified FGF that has been modified at position 12, 83, and 134 is further modified at position 137 with, e.g., Val, Ile, Ser, Thr, Cys, Ala or another amino acid. In some embodiments, the modified FGF that has been modified at position 12, 83, and 134 is further modified at position 9 and position 136 with, e.g., Val, Ile, Ser, Thr, Cys, Ala or another amino acid. In some embodiments, the modified FGF that has been modified at position 12, 83, and 134 is further modified at position 9 and position 137 with, e.g., Val, Ile, Ser, Thr, Cys, Ala or another amino acid. In some embodiments, the modified FGF that has been modified at position 12, 83, and 134 is further modified at position 10 and position 136 with, e.g., Val, Ile, Ser, Thr, Cys, Ala or another amino acid. In some embodiments, the modified FGF that has been modified at position 12, 83, and 134 is further modified at position 10 and position 137 with, e.g., Val, Ile, Ser, Thr, Cys, Ala or another amino acid. In some embodiments, the modified FGF that has been modified at position 12, 83, and 134 is further modified at position 11 and position 136 with, e.g., Val, Ile, Ser, Thr, Cys, Ala or another amino acid. In some embodiments, the modified FGF that has been modified at position 12, 83, and 134 is further modified at position 11 and position 137 with, e.g., Val, Ile, Ser, Thr, Cys, Ala or another amino acid.

In some embodiments, the modified FGF that has been modified at position 12, 117, and 134 is further modified at position 134 with, e.g., Val, Ile, Ser, Thr, Cys, Ala or another amino acid. In some embodiments, the modified FGF that has been modified at position 12, 117, and 134 is further modified at position 9 with, e.g., Val, Ile, Ser, Thr, Cys, Ala or another amino acid. In some embodiments, the modified FGF that has been modified at position 12, 117, and 134 is further modified at position 10 with, e.g., Val, Ile, Ser, Thr, Cys, Ala or another amino acid. In some embodiments, the modified FGF that has been modified at position 12, 117, and 134 is further modified at position 11 with, e.g., Val, Ile, Ser, Thr, Cys, Ala or another amino acid. In some embodiments, the modified FGF that has been modified at position 12, 117, and 134 is further modified at position 136 with, e.g., Val, Ile, Ser, Thr, Cys, Ala or another amino acid. In some embodiments, the modified FGF that has been modified at position 12, 117, and 134 is further modified at position 137 with, e.g., Val, Ile, Ser, Thr, Cys, Ala or another amino acid. In some embodiments, the modified FGF that has been modified at position 12, 117, and 134 is further modified at position 9 and position 136 with, e.g., Val, Ile, Ser, Thr, Cys, Ala or another amino acid. In some embodiments, the modified FGF that has been modified at position 12, 117, and 134 is further modified at position 9 and position 137 with, e.g., Val, Ile, Ser, Thr, Cys, Ala or another amino acid. In some embodiments, the modified FGF that has been modified at position 12, 117, and 134 is further modified at position 10 and position 136 with, e.g., Val, Ile, Ser, Thr, Cys, Ala or another amino acid. In some embodiments, the modified FGF that has been modified at position 12, 117, and 134 is further modified at position 10 and position 137 with, e.g., Val, Ile, Ser, Thr, Cys, Ala or another amino acid. In some embodiments, the modified FGF that has been modified at position 12, 117, and 134 is further modified at position 11 and position 136 with, e.g., Val, Ile, Ser, Thr, Cys, Ala or another amino acid. In some embodiments, the modified FGF that has been modified at position 12, 117, and 134 is further modified at position 11 and position 137 with, e.g., Val, Ile, Ser, Thr, Cys, Ala or another amino acid.

In some embodiments, the modified FGF that has been modified at position 12, 16, and 117 is further modified at position 134 with, e.g., Val, Ile, Ser, Thr, Cys, Ala or another amino acid. In some embodiments, the modified FGF that has been modified at position 12, 16, and 117 is further modified at position 9 with, e.g., Val, Ile, Ser, Thr, Cys, Ala or another amino acid. In some embodiments, the modified FGF that has been modified at position 12, 16, and 117 is further modified at position 10 with, e.g., Val, Ile, Ser, Thr, Cys, Ala or another amino acid. In some embodiments, the modified FGF that has been modified at position 12, 16, and 117 is further modified at position 11 with, e.g., Val, Ile, Ser, Thr, Cys, Ala or another amino acid. In some embodiments, the modified FGF that has been modified at position 12, 16, and 117 is further modified at position 136 with, e.g., Val, Ile, Ser, Thr, Cys, Ala or another amino acid. In some embodiments, the modified FGF that has been modified at position 12, 16, and 117 is further modified at position 137 with, e.g., Val, Ile, Ser, Thr, Cys, Ala or another amino acid. In some embodiments, the modified FGF that has been modified at position 12, 16, and 117 is further modified at position 9 and position 136 with, e.g., Val, Ile, Ser, Thr, Cys, Ala or another amino acid. In some embodiments, the modified FGF that has been modified at position 12, 16, and 117 is further modified at position 9 and position 137 with, e.g., Val, Ile, Ser, Thr, Cys, Ala or another amino acid. In some embodiments, the modified FGF that has been modified at position 12, 16, and 117 is further modified at position 10 and position 136 with, e.g., Val, Ile, Ser, Thr, Cys, Ala or another amino acid. In some embodiments, the modified FGF that has been modified at position 12, 16, and 117 is further modified at position 10 and position 137 with, e.g., Val, Ile, Ser, Thr, Cys, Ala or another amino acid. In some embodiments, the modified FGF that has been modified at position 12, 16, and 117 is further modified at position 11 and position 136 with, e.g., Val, Ile, Ser, Thr, Cys, Ala or another amino acid. In some embodiments, the modified FGF that has been modified at position 12, 16, and 117 is further modified at position 11 and position 137 with, e.g., Val, Ile, Ser, Thr, Cys, Ala or another amino acid.

In some embodiments, the modified FGF that has been modified at position 12, 16, and 134 is further modified at position 134 with, e.g., Val, Ile, Ser, Thr, Cys, Ala or another amino acid. In some embodiments, the modified FGF that has been modified at position 12, 16, and 134 is further modified at position 9 with, e.g., Val, Ile, Ser, Thr, Cys, Ala or another amino acid. In some embodiments, the modified FGF that has been modified at position 12, 16, and 134 is further modified at position 10 with, e.g., Val, Ile, Ser, Thr, Cys, Ala or another amino acid. In some embodiments, the modified FGF that has been modified at position 12, 16, and 134 is further modified at position 11 with, e.g., Val, Ile, Ser, Thr, Cys, Ala or another amino acid. In some embodiments, the modified FGF that has been modified at position 12, 16, and 134 is further modified at position 136 with, e.g., Val, Ile, Ser, Thr, Cys, Ala or another amino acid. In some embodiments, the modified FGF that has been modified at position 12, 16, and 134 is further modified at position 137 with, e.g., Val, Ile, Ser, Thr, Cys, Ala or another amino acid. In some embodiments, the modified FGF that has been modified at position 12, 16, and 134 is further modified at position 9 and position 136 with, e.g., Val, Ile, Ser, Thr, Cys, Ala or another amino acid. In some embodiments, the modified FGF that has been modified at position 12, 16, and 134 is further modified at position 9 and position 137 with, e.g., Val, Ile, Ser, Thr, Cys, Ala or another amino acid. In some embodiments, the modified FGF that has been modified at position 12, 16, and 134 is further modified at position 10 and position 136 with, e.g., Val, Ile, Ser, Thr, Cys, Ala or another amino acid. In some embodiments, the modified FGF that has been modified at position 12, 16, and 134 is further modified at position 10 and position 137 with, e.g., Val, Ile, Ser, Thr, Cys, Ala or another amino acid. In some embodiments, the modified FGF that has been modified at position 12, 16, and 134 is further modified at position 11 and position 136 with, e.g., Val, Ile, Ser, Thr, Cys, Ala or another amino acid. In some embodiments, the modified FGF that has been modified at position 12, 16, and 134 is further modified at position 11 and position 137 with, e.g., Val, Ile, Ser, Thr, Cys, Ala or another amino acid.

In some embodiments, the modified FGF that has been modified at position 12, 16, 83, 117, and 134 is further modified at position 134 with, e.g., Val, Ile, Ser, Thr, Cys, Ala or another amino acid. In some embodiments, the modified FGF that has been modified at position 12, 16, 83, 117, and 134 is further modified at position 9 with, e.g., Val, Ile, Ser, Thr, Cys, Ala or another amino acid. In some embodiments, the modified FGF that has been modified at position 12, 16, 83, 117, and 134 is further modified at position 10 with, e.g., Val, Ile, Ser, Thr, Cys, Ala or another amino acid. In some embodiments, the modified FGF that has been modified at position 12, 16, 83, 117, and 134 is further modified at position 11 with, e.g., Val, Ile, Ser, Thr, Cys, Ala or another amino acid. In some embodiments, the modified FGF that has been modified at position 12, 16, 83, 117, and 134 is further modified at position 136 with, e.g., Val, Ile, Ser, Thr, Cys, Ala or another amino acid. In some embodiments, the modified FGF that has been modified at position 12, 16, 83, 117, and 134 is further modified at position 137 with, e.g., Val, Ile, Ser, Thr, Cys, Ala or another amino acid. In some embodiments, the modified FGF that has been modified at position 12, 16, 83, 117, and 134 is further modified at position 9 and position 136 with, e.g., Val, Ile, Ser, Thr, Cys, Ala or another amino acid. In some embodiments, the modified FGF that has been modified at position 12, 16, 83, 117, and 134 is further modified at position 9 and position 137 with, e.g., Val, Ile, Ser, Thr, Cys, Ala or another amino acid. In some embodiments, the modified FGF that has been modified at position 12, 16, 83, 117, and 134 is further modified at position 10 and position 136 with, e.g., Val, Ile, Ser, Thr, Cys, Ala or another amino acid. In some embodiments, the modified FGF that has been modified at position 12, 16, 83, 117, and 134 is further modified at position 10 and position 137 with, e.g., Val, Ile, Ser, Thr, Cys, Ala or another amino acid. In some embodiments, the modified FGF that has been modified at position 12, 16, 83, 117, and 134 is further modified at position 11 and position 136 with, e.g., Val, Ile, Ser, Thr, Cys, Ala or another amino acid. In some embodiments, the modified FGF that has been modified at position 12, 16, 83, 117, and 134 is further modified at position 11 and position 137 with, e.g., Val, Ile, Ser, Thr, Cys, Ala or another amino acid.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1. In some embodiments, the modified FGF comprises the wild-type human FGF-1 sequence with about 1, about 2, about 3, about 4, about 5, about 6, about 7, about 8, about 9, about 10, about 11, about 12, about 13, about 14, or about 15 residues deleted from the N-terminus. In some embodiments, the modified FGF comprises the wild-type human FGF-1 sequence with about 1, about 2, about 3, about 4, about 5, about 6, about 7, about 8, about 9, about 10, about 11, about 12, about 13, about 14, or about 15 residues deleted from the C-terminus. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109. In some embodiments, the modified FGF comprises the sequence of SEQ ID NO: 109 with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 12 with, for example, the mutation Lys12Val. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 12 with, for example, the mutation Lys12Val. In some embodiments, the modified FGF comprises the wild-type human FGF-1 sequence with a mutation at position 12, for example the mutation Lys12Val, and with about 1, about 2, about 3, about 4, about 5, about 6, about 7, about 8, about 9, about 10, about 11, about 12, about 13, about 14, or about 15 residues deleted from the N-terminus. In some embodiments, the modified FGF comprises the sequence of SEQ ID NO: 109 with a mutation at position 12, for example the mutation Lys12Val, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 12 with, for example, the mutation Lys12Ile. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 12 with, for example, the mutation Lys12Ile. In some embodiments, the modified FGF comprises the wild-type human FGF-1 sequence with a mutation at position 12, for example the mutation Lys12Ile, and with about 1, about 2, about 3, about 4, about 5, about 6, about 7, about 8, about 9, about 10, about 11, about 12, about 13, about 14, or about 15 residues deleted from the N-terminus. In some embodiments, the modified FGF comprises the sequence of SEQ ID NO: 109 with a mutation at position 12, for example the mutation Lys12Ile, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 16 with, for example, the mutation Cys16Ala. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 16 with, for example, the mutation Cys16Ala. In some embodiments, the modified FGF comprises the wild-type human FGF-1 sequence with a mutation at position 12, for example the mutation Cys16Ala, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 12, for example the mutation Cys16Ala, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 83 with, for example, the mutation Cys83Ala. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 83 with, for example, the mutation Cys83Ala. In some embodiments, the modified FGF comprises the wild-type human FGF-1 sequence with a mutation at position 83, for example the mutation Cys83Ala, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 83, for example the mutation Cys83Ala, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 117 with, for example, the mutation Cys117Val. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 117 with, for example, the mutation Cys117Val. In some embodiments, the modified FGF comprises the wild-type human FGF-1 sequence with a mutation at position 12, for example the mutation Cys117Val, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 12, for example the mutation Cys117Val, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 117 with, for example, the mutation Cys117Ile. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 117 with, for example, the mutation Cys117Ile. In some embodiments, the modified FGF comprises the wild-type human FGF-1 sequence with a mutation at position 12, for example the mutation Cys117Ile, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 12, for example the mutation Cys117Ile, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 134 with, for example, the mutation Pro134Val. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 134 with, for example, the mutation Pro134Val. In some embodiments, the modified FGF comprises the wild-type human FGF-1 sequence with a mutation at position 12, for example the mutation Pro134Val, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 12, for example the mutation Pro134Val, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at position 134 with, for example, the mutation Pro134Ile. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at position 134 with, for example, the mutation Pro134Ile. In some embodiments, the modified FGF comprises the wild-type human FGF-1 sequence with a mutation at position 12, for example the mutation Pro134Ile, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation at position 12, for example the mutation Pro134Ile, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more of positions 9, 10, and 11 with, for example, the mutation Lys9Cys, Lys10Cys, and Pro11Cys, respectively. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at one or more of positions 9, 10, and 11 with, for example, the mutation Lys9Cys, Lys10Cys, and Pro11Cys, respectively. In some embodiments, the modified FGF comprises the wild-type human FGF-1 sequence with a mutation at one or more of positions 9, 10, and 11 with, for example, the mutation Lys9Cys, Lys10Cys, and Pro11Cys, respectively, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation one or more of positions 9, 10, and 11 with, for example, the mutation Lys9Cys, Lys10Cys, and Pro11Cys, respectively, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more of positions 136 and 137 with, for example, the mutation Pro136Cys and Val137Cys, respectively. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at one or more of positions 136 and 137 with, for example, the mutation Pro136Cys and Val137Cys, respectively. In some embodiments, the modified FGF comprises the wild-type human FGF-1 sequence with a mutation at one or more of positions 136 and 137 with, for example, the mutation Pro136Cys and Val137Cys, respectively, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with a mutation one or more of positions 136 and 137 with, for example, the mutation Pro136Cys and Val137Cys, respectively, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 12, 117, and 134 with, for example, the mutations Lys12Val, Cys117Val, and Pro134Val. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at one or more positions 12, 117, and 134 with, for example, the mutations Lys12Val, Cys117Val, and Pro134Val. In some embodiments, the modified FGF comprises the wild-type human FGF-1 sequence with a mutation at positions 12, 117 and 134, for example the mutations Lys12Val, Cys117Val, and Pro134Val, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 12, 117 and 134, for example the mutations Lys12Val, Cys117Val, and Pro134Val, and with about 1, about 2, about 3, about 4, about 5, about 6, about 7, about 8, about 9, or about 10, about 11, about 12, about 13, about 14, or about 15 residues from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 12, 117, and 134 with, for example, the mutations Lys12Ile, Cys117Ile, and Pro134Ile. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 mutated at one or more positions 12, 117, and 134 with, for example, the mutations Lys12Ile, Cys117Ile, and Pro134Ile. In some embodiments, the modified FGF comprises the wild-type human FGF-1 sequence with a mutation at positions 12, 117 and 134, for example the mutations Lys12Ile, Cys117Ile, and Pro134Ile, and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 12, 117 and 134, for example the mutations Lys12Ile, Cys117Ile, and Pro134Ile, and with about 1, about 2, about 3, about 4, about 5, about 6, about 7, about 8, about 9, or about 10, about 11, about 12, about 13, about 14, or about 15 residues from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 12, 16, 83, 117, and 134 with, for example, the mutations Lys12Val, Cys16Ala, Cys83Ala, Cys117Val, Pro134Val. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 12, 16, 83, 117, and 134 with, for example, the mutations Lys12Val, Cys16Ala, Cys83Ala, Cys117Val, Pro134Val. In some embodiments, the modified FGF comprises the wild-type human FGF-1 sequence with a mutation at positions 12, 16, 83, 117, and 134 for example the mutations Lys12Val, Cys16Ala, Cys83Ala, Cys117Val, Pro134Val and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 12, 16, 83, 117, and 134 for example the mutations Lys12Val, Cys16Ala, Cys83Ala, Cys117Val, Pro134Val, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 12, 16, 83, 117, and 134 with, for example, the mutations Lys12Ile, Cys16Ala, Cys83Ala, Cys117Ile, Pro134Ile. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 12, 16, 83, 117, and 134 with, for example, the mutations Lys12Ile, Cys16Ala, Cys83Ala, Cys117Ile, Pro134Ile. In some embodiments, the modified FGF comprises the wild-type human FGF-1 sequence with a mutation at positions 12, 16, 83, 117, and 134 for example the mutations Lys12Ile, Cys16Ala, Cys83Ala, Cys117Ile, Pro134Ile and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 12, 16, 83, 117, and 134 for example the mutations Lys12Ile, Cys16Ala, Cys83Ala, Cys117Ile, Pro134Ile, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 9, 12, 16, 83, 117, 134 and 136 with, for example, the mutations Lys9Cys, Lys12Val, Cys16Ala, Cys83Ala, Cys117Val, Pro134Val, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 9, 12, 16, 83, 117, 134 and 136 with, for example, the mutations Lys9Cys, Lys12Val, Cys16Ala, Cys83Ala, Cys117Val, Pro134Val, and Pro136Cys. In some embodiments, the modified FGF comprises the wild-type human FGF-1 sequence with a mutation at positions 9, 12, 16, 83, 117, 134, and 136 for example the mutations Lys9Cys, Lys12Val, Cys16Ala, Cys83Ala, Cys117Val, Pro134Val, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 9, 12, 16, 83, 117, 134, and 136 for example the mutations Lys9Cys, Lys12Val, Cys16Ala, Cys83Ala, Cys117Val, Pro134Val, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 9, 12, 16, 83, 117, 134 and 137 with, for example, the mutations Lys9Cys, Lys12Val, Cys16Ala, Cys83Ala, Cys117Val, Pro134Val, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 9, 12, 16, 83, 117, 134 and 137 with, for example, the mutations Lys9Cys, Lys12Val, Cys16Ala, Cys83Ala, Cys117Val, Pro134Val, and Val137Cys. In some embodiments, the modified FGF comprises the wild-type human FGF-1 sequence with a mutation at positions 9, 12, 16, 83, 117, 134, and 137 for example the mutations Lys9Cys, Lys12Val, Cys16Ala, Cys83Ala, Cys117Val, Pro134Val, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 9, 12, 16, 83, 117, 134, and 137 for example the mutations Lys9Cys, Lys12Val, Cys16Ala, Cys83Ala, Cys117Val, Pro134Val, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 9, 12, 16, 83, 117, 134 and 136 with, for example, the mutations Lys9Cys, Lys12Ile, Cys16Ala, Cys83Ala, Cys117Ile, Pro134Ile, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 9, 12, 16, 83, 117, 134 and 136 with, for example, the mutations Lys9Cys, Lys12Ile, Cys16Ala, Cys83Ala, Cys117Ile, Pro134Ile, and Pro136Cys. In some embodiments, the modified FGF comprises the wild-type human FGF-1 sequence with a mutation at positions 9, 12, 16, 83, 117, 134, and 136 for example the mutations Lys9Cys, Lys12Ile, Cys16Ala, Cys83Ala, Cys117Ile, Pro134Ile, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 9, 12, 16, 83, 117, 134, and 136 for example the mutations Lys9Cys, Lys12Ile, Cys16Ala, Cys83Ala, Cys117Ile, Pro134Ile, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 9, 12, 16, 83, 117, 134 and 137 with, for example, the mutations Lys9Cys, Lys12Ile, Cys16Ala, Cys83Ala, Cys117Ile, Pro134Ile, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 9, 12, 16, 83, 117, 134 and 137 with, for example, the mutations Lys9Cys, Lys12Ile, Cys16Ala, Cys83Ala, Cys117Ile, Pro134Ile, and Val137Cys. In some embodiments, the modified FGF comprises the wild-type human FGF-1 sequence with a mutation at positions 9, 12, 16, 83, 117, 134, and 137 for example the mutations Lys9Cys, Lys12Ile, Cys16Ala, Cys83Ala, Cys117Ile, Pro134Ile, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 9, 12, 16, 83, 117, 134, and 137 for example the mutations Lys9Cys, Lys12Ile, Cys16Ala, Cys83Ala, Cys117Ile, Pro134Ile, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 10, 12, 16, 83, 117, 134 and 136 with, for example, the mutations Lys10Cys, Lys12Val, Cys16Ala, Cys83Ala, Cys117Val, Pro134Val, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 10, 12, 16, 83, 117, 134 and 136 with, for example, the mutations Lys10Cys, Lys12Val, Cys16Ala, Cys83Ala, Cys117Val, Pro134Val, and Pro136Cys. In some embodiments, the modified FGF comprises the wild-type human FGF-1 sequence with a mutation at positions 10, 12, 16, 83, 117, 134, and 136 for example the mutations Lys10Cys, Lys12Val, Cys16Ala, Cys83Ala, Cys117Val, Pro134Val, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 10, 12, 16, 83, 117, 134, and 136 for example the mutations Lys10Cys, Lys12Val, Cys16Ala, Cys83Ala, Cys117Val, Pro134Val, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 10, 12, 16, 83, 117, 134 and 137 with, for example, the mutations Lys10Cys, Lys12Val, Cys16Ala, Cys83Ala, Cys117Val, Pro134Val, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 10, 12, 16, 83, 117, 134 and 137 with, for example, the mutations Lys10Cys, Lys12Val, Cys16Ala, Cys83Ala, Cys117Val, Pro134Val, and Val137Cys. In some embodiments, the modified FGF comprises the wild-type human FGF-1 sequence with a mutation at positions 10, 12, 16, 83, 117, 134, and 137 for example the mutations Lys10Cys, Lys12Val, Cys16Ala, Cys83Ala, Cys117Val, Pro134Val, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 10, 12, 16, 83, 117, 134, and 137 for example the mutations Lys10Cys, Lys12Val, Cys16Ala, Cys83Ala, Cys117Val, Pro134Val, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 10, 12, 16, 83, 117, 134 and 136 with, for example, the mutations Lys10Cys, Lys12Ile, Cys16Ala, Cys83Ala, Cys117Ile, Pro134Ile, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 10, 12, 16, 83, 117, 134 and 136 with, for example, the mutations Lys10Cys, Lys12Ile, Cys16Ala, Cys83Ala, Cys117Ile, Pro134Ile, and Pro136Cys. In some embodiments, the modified FGF comprises the wild-type human FGF-1 sequence with a mutation at positions 10, 12, 16, 83, 117, 134, and 136 for example the mutations Lys10Cys, Lys12Ile, Cys16Ala, Cys83Ala, Cys117Ile, Pro134Ile, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 10, 12, 16, 83, 117, 134, and 136 for example the mutations Lys10Cys, Lys12Ile, Cys16Ala, Cys83Ala, Cys117Ile, Pro134Ile, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 10, 12, 16, 83, 117, 134 and 137 with, for example, the mutations Lys10Cys, Lys12Ile, Cys16Ala, Cys83Ala, Cys117Ile, Pro134Ile, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 10, 12, 16, 83, 117, 134 and 137 with, for example, the mutations Lys10Cys, Lys12Ile, Cys16Ala, Cys83Ala, Cys117Ile, Pro134Ile, and Val137Cys. In some embodiments, the modified FGF comprises the wild-type human FGF-1 sequence with a mutation at positions 10, 12, 16, 83, 117, 134, and 137 for example the mutations Lys10Cys, Lys12Ile, Cys16Ala, Cys83Ala, Cys117Ile, Pro134Ile, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 10, 12, 16, 83, 117, 134, and 137 for example the mutations Lys10Cys, Lys12Ile, Cys16Ala, Cys83Ala, Cys117Ile, Pro134Ile, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 11, 12, 16, 83, 117, 134 and 136 with, for example, the mutations Pro11Cys, Lys12Val, Cys16Ala, Cys83Ala, Cys117Val, Pro134Val, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 11, 12, 16, 83, 117, 134 and 136 with, for example, the mutations Pro11Cys, Lys12Val, Cys16Ala, Cys83Ala, Cys117Val, Pro134Val, and Pro136Cys. In some embodiments, the modified FGF comprises the wild-type human FGF-1 sequence with a mutation at positions 11, 12, 16, 83, 117, 134, and 136 for example the mutations Pro11Cys, Lys12Val, Cys16Ala, Cys83Ala, Cys117Val, Pro134Val, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 11, 12, 16, 83, 117, 134, and 136 for example the mutations Pro11Cys, Lys12Val, Cys16Ala, Cys83Ala, Cys117Val, Pro134Val, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 11, 12, 16, 83, 117, 134 and 137 with, for example, the mutations Pro11Cys, Lys12Val, Cys16Ala, Cys83Ala, Cys117Val, Pro134Val, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 11, 12, 16, 83, 117, 134 and 137 with, for example, the mutations Pro11Cys, Lys12Val, Cys16Ala, Cys83Ala, Cys117Val, Pro134Val, and Val137Cys. In some embodiments, the modified FGF comprises the wild-type human FGF-1 sequence with a mutation at positions 11, 12, 16, 83, 117, 134, and 137 for example the mutations Pro11Cys, Lys12Val, Cys16Ala, Cys83Ala, Cys117Val, Pro134Val, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 11, 12, 16, 83, 117, 134, and 137 for example the mutations Pro11Cys, Lys12Val, Cys16Ala, Cys83Ala, Cys117Val, Pro134Val, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 11, 12, 16, 83, 117, 134 and 136 with, for example, the mutations Pro11Cys, Lys12Ile, Cys16Ala, Cys83Ala, Cys117Ile, Pro134Ile, and Pro136Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more 11, 12, 16, 83, 117, 134 and 136 with, for example, the mutations Pro11Cys, Lys12Ile, Cys16Ala, Cys83Ala, Cys117Ile, Pro134Ile, and Pro136Cys. In some embodiments, the modified FGF comprises the wild-type human FGF-1 sequence with a mutation at positions 11, 12, 16, 83, 117, 134, and 136 for example the mutations Pro11Cys, Lys12Ile, Cys16Ala, Cys83Ala, Cys117Ile, Pro134Ile, and Pro136Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 11, 12, 16, 83, 117, 134, and 136 for example the mutations Pro11Cys, Lys12Ile, Cys16Ala, Cys83Ala, Cys117Ile, Pro134Ile, and Pro136Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to wild-type human FGF-1 mutated at one or more positions 11, 12, 16, 83, 117, 134 and 137 with, for example, the mutations Pro11Cys, Lys12Ile, Cys16Ala, Cys83Ala, Cys117Ile, Pro134Ile, and Val137Cys. In some embodiments, the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 109 at one or more positions 11, 12, 16, 83, 117, 134 and 137 with, for example, the mutations Pro11Cys, Lys12Ile, Cys16Ala, Cys83Ala, Cys117Ile, Pro134Ile, and Val137Cys. In some embodiments, the modified FGF comprises the wild-type human FGF-1 sequence with a mutation at positions 11, 12, 16, 83, 117, 134, and 137 for example the mutations Pro11Cys, Lys12Ile, Cys16Ala, Cys83Ala, Cys117Ile, Pro134Ile, and Val137Cys and with about 1, about 2, about 3, about 4, about 5, about 6, about 7, about 8, about 9, or about 10, about 11, about 12, about 13, about 14, or about 15 residues deleted from the N-terminus. In some embodiments, the modified FGF comprises the sequence of SEQ ID NO: 109 with mutations at position 11, 12, 16, 83, 117, 134, and 137 for example the mutations Pro11Cys, Lys12Ile, Cys16Ala, Cys83Ala, Cys117Ile, Pro134Ile, and Val137Cys, and with about 1, about 2, about 3, about 4, about 5, about 6, about 7, about 8, about 9, or about 10, about 11, about 12, about 13, about 14, or about 15 residues deleted from the C-terminus.

In some embodiments, the modified FGF comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108.

In some embodiments, a modified FGF comprises the substitutions K12V C117V, P134V, C16A, C83A, K9C, P136C, truncation of the first five residues of FGF-1, and truncation of the last three residues of FGF-1. In some embodiments, a modified FGF comprises the sequence of SEQ ID NO: 97, wherein the struck-through residues denote truncated residues.

In some embodiments, a modified FGF comprises the substitutions K12V C117V, P134V, C16A, C83A, K9C, V137C, truncation of the first five residues of FGF-1, and truncation of the last three residues of FGF-1. In some embodiments, a modified FGF comprises the sequence of SEQ ID NO: 98, wherein the struck-through residues denote truncated residues.

In some embodiments, a modified FGF comprises the substitutions K12V C117V, P134V, C16A, C83A, K10C, P136C, truncation of the first five residues of FGF-1, and truncation of the last three residues of FGF-1. In some embodiments, a modified FGF comprises the sequence of SEQ ID NO: 99, wherein the struck-through residues denote truncated residues.

In some embodiments, a modified FGF comprises the substitutions K12V C117V, P134V, C16A, C83A, K10C, V137C, truncation of the first five residues of FGF-1, and truncation of the last three residues of FGF-1. In some embodiments, a modified FGF comprises the sequence of SEQ ID NO: 100, wherein the struck-through residues denote truncated residues.

In some embodiments, a modified FGF comprises the substitutions K12V C117V, P134V, C16A, C83A, P11C, P136C, truncation of the first five residues of FGF-1, and truncation of the last three residues of FGF-1. In some embodiments, a modified FGF comprises the sequence of SEQ ID NO: 101, wherein the struck-through residues denote truncated residues.

In some embodiments, a modified FGF comprises the substitutions K12V C117V, P134V, C16A, C83A, P11C, V137C, truncation of the first five residues of FGF-1, and truncation of the last three residues of FGF-1. In some embodiments, a modified FGF comprises the sequence of SEQ ID NO: 102, wherein the struck-through residues denote truncated residues.

In some embodiments, a modified FGF comprises the substitutions K12I, C117I, P134I, C16A, C83A, K9C, P136C, truncation of the first five residues of FGF-1, and truncation of the last three residues of FGF-1. In some embodiments, a modified FGF comprises the sequence of SEQ ID NO: 103, wherein the struck-through residues denote truncated residues.

In some embodiments, a modified FGF comprises the substitutions K12I, C117I, P134I, C16A, C83A, K9C, V137C, truncation of the first five residues of FGF-1, and truncation of the last three residues of FGF-1. In some embodiments, a modified FGF comprises the sequence of SEQ ID NO: 104, wherein the struck-through residues denote truncated residues.

In some embodiments, a modified FGF comprises the substitutions K12I, C117I, P134I, C16A, C83A, K10C, P136C, truncation of the first five residues of FGF-1, and truncation of the last three residues of FGF-1. In some embodiments, a modified FGF comprises the sequence of SEQ ID NO: 105, wherein the struck-through residues denote truncated residues.

In some embodiments, a modified FGF comprises the substitutions K12I, C117I, P134I, C16A, C83A, K10C, V137C, truncation of the first five residues of FGF-1, and truncation of the last three residues of FGF-1. In some embodiments, a modified FGF comprises the sequence of SEQ ID NO: 106, wherein the struck-through residues denote truncated residues.

In some embodiments, a modified FGF comprises the substitutions K12I, C117I, P134I, C16A, C83A, P11, P136C, truncation of the first five residues of FGF-1, and truncation of the last three residues of FGF-1. In some embodiments, a modified FGF comprises the sequence of SEQ ID NO: 107, wherein the struck-through residues denote truncated residues.

In some embodiments, a modified FGF comprises the substitutions K12I, C117I, P134I, C16A, C83A, P11C, V137C, truncation of the first five residues of FGF-1, and truncation of the last three residues of FGF-1. In some embodiments, a modified FGF comprises the sequence of SEQ ID NO: 108, wherein the struck-through residues denote truncated residues.

In some embodiments, the signal peptide is removed from the modified FGF. In some embodiments, the signal peptide is not removed from the modified FGF.

In some embodiments, the modified FGFs or compositions described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

The modified FGFs described herein may be labeled isotopically (e.g. with a radioisotope) or by other means, including, but not limited to, the use of chromophores or fluorescent moieties, bioluminescent labels, photoactivatable or chemiluminescent labels.

In some embodiments, the synthesis of modified FGFFs described herein is accomplished using means described in the art, using the methods described herein, or by a combination thereof. As a non-limiting example, the FGF proteins described herein are, in some embodiments, synthesized using an N-terminal extension comprising a poly His sequence (for metal ion affinity chromatography) with an enterokinase recognition motif (DDDDK) inserted before the glycine (position 6 in the 140 sequence of hFGF1 (SEQ ID NO: 109). In some embodiments, following expression and initial purification on a nickel column, the enterokinase releases the modified 132 amino acid FGF-1, which is then recovered by a second metal chromatography. In some embodiments, greater recovery is achieved by forming mixed disulfide derivatives and initiating disulfide interchange with traces of thiol compounds.

```
Sequences
K12V/C117V/P134V
SEQ ID NO: 1
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/K9C
SEQ ID NO: 2
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/K10C
SEQ ID NO: 3
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/P11C
SEQ ID NO: 4
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/K9C/P136C
SEQ ID NO: 5
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/K9C/V137C
SEQ ID NO: 6
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/K10C/P136C
SEQ ID NO: 7
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/K10C/V137C
SEQ ID NO: 8
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/P11C/P136C
SEQ ID NO: 9
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/P11C/V137C
SEQ ID NO: 10
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/C16A
SEQ ID NO: 11
1 10 20 30 40
FNLPPGNYKK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/C16A/K9C
SEQ ID NO: 12
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/C16A/K10C
SEQ ID NO: 13
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/C16A/P11C
SEQ ID NO: 14
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPVSSD
K12V/C117V/P134V/C16A/P136C
SEQ ID NO: 15
1 10 20 30 40
FNLPPGNYKK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/C16A/V137C
SEQ ID NO: 16
1 10 20 30 40
FNLPPGNYKK PVLLYPSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/C16A/K9C/P136C
SEQ ID NO: 17
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/C16A/K9C/V137C
SEQ ID NO: 18
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/C16A/K10C/P136C
SEQ ID NO: 19
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/C16A/K10C/V137C
SEQ ID NO: 20
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/C16A/P11C/P136C
SEQ ID NO: 21
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/C16A/P11C/V137C
SEQ ID NO: 22
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12V/C117V/P134V/C83A
SEQ ID NO: 23
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C83A/K9C
SEQ ID NO: 24
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C83A/K10C
SEQ ID NO: 25
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C83A/P11C
SEQ ID NO: 26
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C83A/P136C
SEQ ID NO: 27
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C83A/V137C
SEQ ID NO: 28
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C83A/K9C/P136C
SEQ ID NO: 29
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C83A/K9C/V137C
SEQ ID NO: 30
1 10 20 30 40
FNLPPGNYCK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C83A/K10C/P136C
SEQ ID NO: 31
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C83A/K10C/V137C
SEQ ID NO: 32
1 10 20 30 40
FNLPPGNYKC PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C83A/P11C/P136C
SEQ ID NO: 33
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C83A/P11C/V137C
SEQ ID NO: 34
1 10 20 30 40
FNLPPGNYKK CVLLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C16A/C83A
SEQ ID NO: 35
1 10 20 30 40
FNLPPGNYKK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C16A/C83A/K9C
SEQ ID NO: 36
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C16A/C83A/K10C
SEQ ID NO: 37
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C16A/C83A/P11C
SEQ ID NO: 38
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPVSSD
K12V/C117V/P134V/C16A/C83A/P136C
SEQ ID NO: 39
1 10 20 30 40
FNLPPGNYKK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C16A/C83A/V137C
SEQ ID NO: 40
1 10 20 30 40
FNLPPGNYKK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C16A/C83A/K9C/P136C
SEQ ID NO: 41
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C16A/C83A/K9C/V137C
SEQ ID NO: 42
1 10 20 30 40
FNLPPGNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C16A/C83A/K10C/P136C
SEQ ID NO: 43
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C16A/C83A/K10C/V137C
SEQ ID NO: 44
1 10 20 30 40
FNLPPGNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12V/C117V/P134V/C16A/C83A/P11C/P136C
SEQ ID NO: 45
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCVSSD
K12V/C117V/P134V/C16A/C83A/P11C/V137C
SEQ ID NO: 46
1 10 20 30 40
FNLPPGNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPCSSD
K12I/C117I/P134I
SEQ ID NO: 47
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/K9C
SEQ ID NO: 48
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/K10C
SEQ ID NO: 49
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/P11C
SEQ ID NO: 50
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/K9C/P136C
SEQ ID NO: 51
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/K9C/V137C
SEQ ID NO: 52
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/K10C/P136C
SEQ ID NO: 53
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/K10C/V137C
SEQ ID NO: 54
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/P11C/P136C
SEQ ID NO: 55
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/P11C/V137C
SEQ ID NO: 56
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/C16A
SEQ ID NO: 57
1 10 20 30 40
FNLPPGNYKK PILLYSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/C16A/K9C
SEQ ID NO: 58
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/C16A/K10C
SEQ ID NO: 59
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/C16A/P11C
SEQ ID NO: 60
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPVSSD
K12I/C117I/P134I/C16A/P136C
SEQ ID NO: 61
1 10 20 30 40
FNLPPGNYKK PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/C16A/V137C
SEQ ID NO: 62
1 10 20 30 40
FNLPPGNYKK PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/C16A/K9C/P136C
SEQ ID NO: 63
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/C16A/K9C/V137C
SEQ ID NO: 64
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/C16A/K10C/P136C
SEQ ID NO: 65
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/C16A/K10C/V137C
SEQ ID NO: 66
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/C16A/P11C/P136C
SEQ ID NO: 67
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/C16A/P11C/V137C
SEQ ID NO: 68
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12I/C117I/P134I/C83A
SEQ ID NO: 69
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C83A/K9C
SEQ ID NO: 70
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C83A/K10C
SEQ ID NO: 71
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C83A/P11C
SEQ ID NO: 72
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLTLPVSSD
K12I/C117I/P134I/C83A/P136C
SEQ ID NO: 73
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSTKRG
              130 140
PRTHYGQKAI LFLILCVSSD
K12I/C117I/P134I/C83A/V137C
SEQ ID NO: 74
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C83A/K9C/P136C
SEQ ID NO: 75
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C83A/K9C/V137C
SEQ ID NO: 76
1 10 20 30 40
FNLPPGNYCK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C83A/K10C/P136C
SEQ ID NO: 77
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C83A/K10C/V137C
SEQ ID NO: 78
1 10 20 30 40
FNLPPGNYKC PILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C83A/P11C/P136C
SEQ ID NO: 79
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C83A/P11C/V137C
SEQ ID NO: 80
1 10 20 30 40
FNLPPGNYKK CILLYCSNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C16A/C83A
SEQ ID NO: 81
1 10 20 30 40
FNLPPGNYKK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C16A/C83A/K9C
SEQ ID NO: 82
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C16A/C83A/K10C
SEQ ID NO: 83
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C16A/C83A/P11C
SEQ ID NO: 84
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPVSSD
K12I/C117I/P134I/C16A/C83A/P136C
SEQ ID NO: 85
1 10 20 30 40
FNLPPGNYKK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C16A/C83A/V137C
SEQ ID NO: 86
1 10 20 30 40
FNLPPGNYKK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C16A/C83A/K9C/P136C
SEQ ID NO: 87
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C16A/C83A/K9C/V137C
SEQ ID NO: 88
1 10 20 30 40
FNLPPGNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C16A/C83A/K10C/P136C
SEQ ID NO: 89
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C16A/C83A/K10C/V137C
SEQ ID NO: 90
1 10 20 30 40
FNLPPGNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12I/C117I/P134I/C16A/C83A/P11C/P136C
SEQ ID NO: 91
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCVSSD
K12I/C117I/P134I/C16A/C83A/P11C/V137C
SEQ ID NO: 92
1 10 20 30 40
FNLPPGNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPCSSD
K12V/C117V/P134V/P136C
SEQ ID NO: 93
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLCVSSD
K12V/C117V/P134V/V137C
SEQ ID NO: 94
1 10 20 30 40
FNLPPGNYKK PVLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSVKRG
              130 140
PRTHYGQKAI LFLVLPCSSD
K12I/C117I/P134I/P136C
SEQ ID NO: 95
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILCVSSD
K12I/C117I/P134I/V137C
SEQ ID NO: 96
1 10 20 30 40
FNLPPGNYKK PILLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSIKRG
              130 140
PRTHYGQKAI LFLILPCSSD
K12V/C117V/P134V/C16A/C83A/K9C/P136C
SEQ ID NO: 97
1 10 20 30 40 [symbol]
GNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCV [symbol]
K12V/C117V/P134V/C16A/C83A/K9C/V137C
SEQ ID NO: 98
1 10 20 30 40 [symbol]
GNYCK PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPC [symbol]
K12V/C117V/P134V/C16A/C83A/K10C/P136C
SEQ ID NO: 99
1 10 20 30 40 [symbol]
GNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLCV [symbol]
K12V/C117V/P134V/C16A/C83A/K10C/V137C
SEQ ID NO: 100
1 10 20 30 40 [symbol]
GNYKC PVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPC [symbol]
K12V/C117V/P134V/C16A/C83A/P11C/P136C
SEQ ID NO: 101
1 10 20 30 40 [symbol]
GNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVCV [symbol]
K12V/C117V/P134V/C16A/C83A/P11C/V137C
SEQ ID NO: 102
1 10 20 30 40 [symbol]
GNYKK CVLLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSVKRG
              130 140
PRTHYGQKAI LFLVLPC [symbol]
K12I/C117I/P134I/C16A/C83A/K9C/P136C
SEQ ID NO: 103
1 10 20 30 40 [symbol]
GNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCV [symbol]
K12I/C117I/P134I/C16A/C83A/K9C/V137C
SEQ ID NO: 104
1 10 20 30 40 [symbol]
GNYCK PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPC [symbol]
K12I/C117I/P134I/C16A/C83A/K10C/P136C
SEQ ID NO: 109
1 10 20 30 40 [symbol]
GNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCV [symbol]
K12I/C117I/P134I/C16A/C83A/K10C/V137C
SEQ ID NO: 106
1 10 20 30 40 [symbol]
GNYKC PILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPC [symbol]
K12I/C117I/P134I/C16A/C83A/P11C/P136C
SEQ ID NO: 107
1 10 20 30 40 [symbol]
GNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILCV [symbol]
K12I/C117I/P134I/C16A/C83A/P11C/V137C
SEQ ID NO: 108
1 10 20 30 40 [symbol]
GNYKK CILLYASNGG HFLRILPDGT VDGTRDRSDQ
                50 60 70 80
HIQLQLSAES VGEVYIKSTE TGQYLAMDTD GLLYGSQTPN
                90 100 110 120
EEALFLERLE ENHYNTYISK KHAEKNWFVG LKKNGSIKRG
              130 140
PRTHYGQKAI LFLILPC [symbol]
Wild Type FGF-1
SEQ ID NO: 109
1 10 20 30 40
FNLPPGNYKK PKLLYCSNGG HFLRILPDGT VDGTRDRSDQ
                50 60 70 80
HIQLQLSAES VGEVYIKSTE TGQYLAMDTD GLLYGSQTPN
                90 100 110 120
EECLFLERLE ENHYNTYISK KHAEKNWFVG LKKNGSCKRG
              130 140
PRTHYGQKAI LFLPLPVSSD
```

For SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, and SEQ ID NO: 108, the struck-through residues denote truncated/deleted residues.

### Methods of Use

Provided herein are modified fibroblast growth factors (FGFs), pharmaceutical compositions and medicaments, and methods of using such modified FGFs to treat diseases, disorders, or conditions. In some embodiments, the modified FGFs described herein are used to treat corneal disease, disorders and conditions, e.g., corneal endothelial dystrophies such as Fuch's dystrophy (FD). In some embodiments, the modified FGFs described herein are used to treat an ocular disease disorder or condition in a mammal. In some embodiments, the modified FGFs described herein are used to treat a retinal disease in a mammal. In some embodiments, the modified FGFs described herein are used to treat a wound, for example a skin wound, in a mammal. In some embodiments, the modified FGFs described herein are used to treat a broken bone in a mammal. In some embodiments, the modified FGFs described herein are used to treat a bone lesion in a mammal. In some embodiments, the modified FGFs described herein are used to rejuvenate or reduce scar formation in a mammal.

Provided herein is a method of treating an ocular disease, disorder or condition in a mammal comprising administering to the mammal a modified fibroblast growth factor (FGF). In some embodiments, the ocular disease, disorder or condition to be treated is a disease, disorder, or condition of the corneal endothelial layer. Diseases, disorders, or conditions of the corneal endothelial layer include, but are not limited to, Fuch's dystrophy, bullous keratopathy, congenital hereditary endothelial dystrophy 1, congenital hereditary endothelial dystrophy 2, posterior polymorphous corneal dystrophy, and dry eye syndromes.

Without being bound by theory, it is believed a solution of a modified FGF injected intracamerally into the aqueous humor of the eye binds to the endothelial surface and especially any areas of the cornea that are not covered by a healthy endothelial layer. The modified FGF stimulates the growth and migration of the endothelial cells. This reduces the corneal edema associated with the endothelial dystrophy and reduces the likelihood for a need for a corneal or endothelial transplant. The action of the modified FGF can occur at a site other than the site of greatest dystrophy (typically at the corneal center) and also results in stimulation of endothelial cells in the corneal periphery and endothelial progenitor pools in the trabecular meshwork (TM).

In some embodiments, the ocular disease, disorder or condition to be treated is a disease, disorder, or condition of the corneal epithelium. Diseases, disorders or conditions of the corneal epithelium include, but are not limited to, dry eye syndromes, inflammatory conditions such as Stevens-Johnson syndrome, and corneal epithelial defects.

In further embodiments, the modified FGFs described herein can be used to treat epithelial basement membrane dystrophy, Meesmann juvenile epithelial corneal dystrophy, gelatinous drop-like corneal dystrophy, Lisch epithelial corneal dystrophy, subepithelial mucinous corneal dystrophy, Reis-Bucklers corneal dystrophy, or Thiel-Behnke dystrophy, and recurrent corneal erosions.

In some embodiments, the ocular condition includes damage to the cornea (e.g., the corneal surface or endothelial layer at the interface of the cornea and aqueous humor) or surgical disruption caused by corneal surgeries, including PRK, LASIK, and any penetrating corneal surgery or keratoplasty.

In some embodiments, the ocular condition includes accidental trauma or chemical or thermal injury to the cornea. In some embodiments, the ocular condition comprises mustard gas keratopathy.

In some embodiments, the ocular disease, disorder or condition to be treated is a disease, disorder, or condition of the corneal stroma. Diseases, disorders or conditions of the corneal stroma include, but are not limited to, keratoconus, lattice corneal dystrophy, granular corneal dystrophy, macular corneal dystrophy, Schnyder crystalline corneal dystrophy, congenital stromal corneal dystrophy, fleck corneal dystrophy, trauma or chemical or thermal injury, or injury secondary to infections such as trachoma.

In further embodiments, the modified FGFs described herein can be applied before, during, or after corneal transplantations procedures (e.g., corneal transplantation or procedures involving Descemet's membrane) that involve disruption of the cornea (e.g., corneal endothelial structure) where acceleration of healing of corneal or ocular surface cells and/or improving the cellular response (e.g, by increasing the viability and/or longevity of the transplanted cells) to insult would result in a therapeutic benefit.

In additional embodiments, the modified FGFs described herein can be used to increase the viability and health of corneal cells or corneal progenitors being prepared for transplantation. Modified FGFs added to the organ culture medium for donated corneas or other donated corneal tissue stimulates the corneal cells and increases the length of time the corneas can be stored before transplantation, as well as increasing the probability that a cornea will have sufficient healthy cells to be useful for transplantation. Also, the modified FGFs can be used in culture media when culturing corneal progenitor cells to stimulate growth of those cells.

Further embodiments relate to methods of modulating the activity of one or more fibroblast growth factor receptors (FGFRs) in a corneal endothelial cell comprising contacting said corneal endothelial cell with a modified FGF. Such methods can be used to increase or stimulate the activity of one or more FGFRs, which can result in increased cell migration and/or cell proliferation.

In some embodiments one or more modified FGFs are used to induce regeneration or tissue healing without EMT or scarring. In some embodiments, modified FGFs described herein FGFs are used to decrease the amount of scarring associated with the healing process. In some embodiments, modified FGFs are applied to surgical incisions to reduce the amount of scar formation. This is particularly useful where the scarring response results in decreased function of the tissue, for example in trabeculectomy procedures where scarring of the tissue leads to blockage of the drainage channel created by the trabeculectomy. This is also useful where scar tissue formation is esthetically undesirable, such as in surgical incisions to the skin.

In some embodiments, pharmaceutical compositions described herein comprising one or more modified FGFs are administered by local injection, placement, catheter delivery, or implantation at a desired site of action in the body of an individual, subject, or patient, such as a tissue or cellular environment at or near a site of a disorder or disease, such as at or near a site of ischemic or hypoxic stress or a site of an injury or wound. In some embodiments, pharmaceutical compositions described herein comprising one or more modified FGFs are administered by local injection, placement, or implantation at or near a site causing an ischemic or hypoxic stress or condition at a different site, such as a site of vessel occlusion. In various embodiments, such a local injection, placement, or implantation of pharmaceutical compositions of the present invention may include any suitable peri- and intra-tissue injections, such as intradermal, intramuscular, intracardiac, subcutaneous, intrathecal, etc. as the case may be. In some embodiments, pharmaceutical compositions described herein comprising one or more modified FGFs are administered by local injection, placement, or implantation at two or more sites at or near a desired site of action.

In some embodiments, for local injection, placement, or implantation, embodiments of pharmaceutical compositions described herein are formulated with a variety of aqueous or non-aqueous solutions, suspensions, emulsions, etc. as described above, such as physiologically compatible buffers including Hank's solution, Ringer's solution, physiological saline buffer, etc. In various embodiments pharmaceutical compositions for local injection, placement, or implantation comprise biocompatible materials or polymers providing sustained release or restricted diffusion as described above. In some embodiments, pharmaceutical compositions for parenteral administration, solutions and suspensions for local or topical administration are freshly prepared or resuspended from a dry preparation of a modified FGF, such as a lyophilized or spray dried preparation, prior to its use.

In some embodiments, pharmaceutical compositions described herein comprising one or more modified FGFs are administered topically, such as at a site of a tissue injury or a wound. In some embodiments, pharmaceutical compositions for topical administration are formulated as a liquid or semi-solid material, such as a gel, paste, putty, ointment, cream, emulsion, patch, etc. as well as other biocompatible materials or polymers. In some embodiments, pharmaceutical compositions for topical administration are formulated as a dry or solid preparation, such as a powders, granules, etc., that are applied directly to a desired site of action. According to some embodiments, pharmaceutical compositions for topical administration are molded into a desired size and shape, such as for placement within or to fill a space at a desired site of administration in the body of an individual, subject, or patient, such as at a site of a tissue injury or wound to promote healing. In some embodiments, pharmaceutical compositions described herein are topically administered at two or more sites at or near a tissue injury or a wound.

In some embodiments, a therapeutically effective amount of a modified FGF protein is an amount effective to achieve a desired result, purpose, or therapeutic benefit, such as an amount effective to prevent, alleviate, ameliorate, treat, etc. the underlying causes and/or symptoms of a condition or disease, such as an ischemic or hypoxic condition or disease or a wound or tissue damage. According to some embodiments, a therapeutically effective amount of a modified FGF is an amount effective to increase blood flow, angiogenesis, and/or vascularization within or to a particular tissue or region of the body of an individual, subject, or patient, such as a tissue or region of the body experiencing ischemia and/or hypoxic conditions. Increased blood flow, angiogenesis, and/or vascularization within or to such a tissue or region of the body, in some embodiments, is determined by a skilled scientist, veterinarian, or physician using any known reagents and pathological or clinical techniques, such as imaging techniques using a contrast dye to detect vasculature, reduction of clinical symptoms associated with an underlying ischemic or hypoxic condition or disease, etc. In some embodiments, a modified FGF is administered to cardiac tissue in a therapeutically effective amount, wherein a therapeutically effective amount is effective to reduce clinical symptoms of coronary artery disease, such as reduction in angina, breathlessness, leg swelling, heart or respiratory rates, edema, fatigue, weakness, etc., or to reduce the risk of a myocardial infarction. In some embodiments, a therapeutically effective amount of a modified FGF is an amount effective to improve the quality and/or rate of healing or repair of a damaged tissue or wound according to known standards and knowledge generally available to a skilled scientist, veterinarian, or physician as the case may be.

Provided herein are methods or improving the quality and/or rate of tissue repair and/or wound healing by administering a modified FGF to an individual, subject, or patient. In some embodiments, a composition comprising a modified FGF is administered by injection, placement, or implantation at, into, onto, or near a site of a wound or tissue damage. In some embodiments, a wound or tissue damage is caused by traumatic injury. In some embodiments, a wound or tissue damage is immunologically mediated. In various embodiments, by administering a modified FGF to a site of a wound or tissue damage, wound healing or tissue repair is promoted or improved by increased growth, proliferation, and/or survival of cells and/or angiogenesis to provide blood flow to the repaired or healed tissue. For example, a composition comprising one or more modified FGFs is administered according to some embodiments to cardiac or brain tissue following myocardial infarction or stroke, respectively, to promote repair, neovascularization, and/or healing of the damaged tissues. In some embodiments, a composition comprising one or more modified FGFs is administered by injection, catheterization, placement, or implantation at, into, onto, or near a site of an incision or tissue damage or removal resulting, at least in part, from a surgical operation to promote healing and repair of the tissue.

### Pharmaceutical Compositions, Methods of Administration, and Dosing

In some embodiments, pharmaceutical compositions comprising a modified FGF described herein are formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a modified FGF with other chemical components, such as carriers, stabilizers, diluents, dispersing agents, suspending agents, thickening agents, and/or excipients, and, optionally, other therapeutic and/or prophylactic ingredients. The pharmaceutical composition facilitates administration of the modified FGF to an organism. In practicing the methods of treatment or use provided herein, therapeutically effective amounts of modified FGFs described herein are administered in a pharmaceutical composition to a mammal having an ocular disease, disorder, or condition to be treated. In some embodiments, the mammal is a human. A therapeutically effective amount can vary widely depending on the severity of the disease, the age and relative health of the subject, the potency of the compound used and other factors. A pharmaceutically acceptable or suitable composition includes an ophthalmologically suitable or acceptable composition.

In some embodiments, a pharmaceutical composition (e.g., for delivery by injection or for application as an eye drop) is in the form of a liquid or solid. A liquid pharmaceutical composition may include, for example, one or more of the following: sterile diluents such as water for injection, saline solution, preferably physiological saline, Ringer's solution, isotonic sodium chloride, phosphate-buffer saline, citrate-buffer saline, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. Physiological saline is commonly used as an excipient, and an injectable pharmaceutical composition or a composition that is delivered ocularly is preferably sterile.

A modified FGF or pharmaceutical composition described herein can be delivered to a subject by any suitable means, including, for example, topically, intraocularly, intracamerally, orally, parenterally, intravenously, intraperitoneally, intranasally (or other delivery methods to the mucous membranes, for example, of the nose, throat, and bronchial tubes), or by local administration to the eye, or by an intraocular or periocular device. Modes of local administration can include, for example, topical application, eye drops, intraocular injection or periocular injection. Periocular injection typically involves injection of the compound under the conjunctiva or into the Tennon's space (beneath the fibrous tissue overlying the eye). Intraocular injection typically involves injection of the modified FGF or pharmaceutical composition into the vitreous human or aqueous humor. In certain embodiments, the administration is non-invasive, such as by topical application or eye drops.

A modified FGF or pharmaceutical composition described herein can be formulated for administration using pharmaceutically acceptable (suitable) carriers or vehicles as well as techniques routinely used in the art. A pharmaceutically acceptable or suitable carrier includes an ophthalmologically suitable or acceptable carrier. A carrier is selected according to the solubility of the particular modified FGF. Suitable ophthalmological compositions and formulations include those that are administrable locally to the eye, such as by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

For injection, the modified FGF or pharmaceutical composition can be provided in an injection grade saline solution, in the form of an injectable liposome solution, slow-release polymer system or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

In some embodiments, the modified FGF or pharmaceutical composition (e.g., an ophthalmic formulation) is administered via microneedles into the cornea (Jiang et al. (2007). Invest Ophthalmol Vis Sci 48(9): 4038-4043). A microneedle array is coated with the modified FGF or pharmaceutical composition and pressed against the cornea such that the microneedles penetrate into the corneal stroma but do not penetrate the entire cornea. It is then removed, and the modified FGF or pharmaceutical composition is left behind in the corneal stroma. This modified FGF or pharmaceutical composition can stimulates the corneal cells to proliferate and migrate, and suppresses the scarring response that the stromal cells normally have.

For delivery of a composition comprising at least one of the modified FGFs described herein via a mucosal route, which includes delivery to the nasal passages, throat, and airways, the composition, in some embodiments, is delivered in the form of an aerosol. In some embodiments, the compound is in a liquid or powder form for intramucosal delivery. For example, the composition is delivered via a pressurized aerosol container with a suitable propellant, such as a hydrocarbon propellant (e.g., propane, butane, isobutene). In some embodiments, the composition is delivered via a non-pressurized delivery system such as a nebulizer or atomizer.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

In some embodiments, the modified FGFs or pharmaceutical compositions described herein are formulated for sustained or slow-release. Such compositions may generally be prepared using well known technology and administered by, for example, periocular, intraocular, rectal, oral or subcutaneous implantation, or by implantation at the desired target site, or by topical application. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained-release formulation depends upon the site of implantation, the rate and expected duration of release, and the nature of the condition to be treated or prevented.

Systemic drug absorption of a drug or composition administered via an ocular route is known to those skilled in the art (see, e.g., Lee et al., Int. J. Pharm. 233:1-18 (2002)). In one embodiment, a compound described herein is delivered by a topical ocular delivery method (see, e.g., Curr. Drug Metab. 4:213-22 (2003)). In some embodiments, the composition is in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used.

In another embodiment, the modified FGF solution or pharmaceutical composition (e.g., an ophthalmic formulation) contains hyaluronic acid, carboxymethyl cellulose, or other polysaccharides that provide increased ocular tolerability, viscosity and osmolality to produce a comfortable ocular solution.

The dose of the modified FGF or pharmaceutical composition comprising at least one of the modified FGFs described herein may differ, depending upon the patient's (e.g., human) condition, that is, stage of the ocular disease, disorder, or condition, general health status, age, and other factors that a person skilled in the medical art will use to determine dose. When the composition is used as eye drops, for example, one to several drops per unit dose, preferably 1 or 2 drops (about 50 μl per 1 drop), are applied about 1 to about 6 times daily.

In some embodiments, pharmaceutical compositions are administered in a manner appropriate to the ocular disease, disorder, or condition to be treated (or prevented) as determined by persons skilled in the medical arts. An appropriate dose and a suitable duration and frequency of administration will be determined by such factors as the condition of the patient, the type and severity of the patient's disease, disorder, or condition, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free, or a lessening of symptom severity). For prophylactic use, a dose should be sufficient to prevent, delay the onset of, or diminish the severity of an ocular disease, disorder, or condition. Optimal doses may generally be determined using experimental models and/or clinical trials. The optimal dose may depend upon the body mass, weight, or blood volume of the patient.

The doses of the modified FGFs or pharmaceutical compositions can be suitably selected depending on the clinical status, condition and age of the subject, dosage form and the like. In the case of eye drops, a modified FGF described herein is administered, for example, from about 1 ng/ml to about 100 mg/ml of the modified FGF one to seven times per week. In the case of intracameral injection, a modified FGF described herein is administered about once per month, about once every two months, about once every three months, about once every four months, about once every five months, about once every six months, about once every seven months, about once every eight months, about once every nine months, about once every ten months, about once every eleven months, or about once every twelve months. In the case of topical administration, a modified FGF described herein is administered, about once per day, about twice per day, about three times per day, about once every other day, about once every three days, about once every four days, about once every five days, about once every six days, or about once per week.

Also provided are methods of manufacturing the modified FGFs and pharmaceutical compositions described herein. In some embodiments, a composition comprising a pharmaceutically acceptable excipient or carrier and at least one of the modified FGFs described herein is prepared by synthesizing the modified FGF according to any one of the methods described herein or practiced in the art and then formulating the compound with a pharmaceutically acceptable carrier. Formulation of the composition will be appropriate and dependent on several factors, including but not limited to, the delivery route, dose, and stability of the compound.

At least one modified FGF described herein can be administered to human or other nonhuman vertebrates. In certain embodiments, the modified FGF is substantially pure, in that it contains less than about 5% or less than about 1%, or less than about 0.1%, of other organic molecules, such as contaminating intermediates or by-products that are created, for example, in one or more of the steps of a synthesis method. In other embodiments, a combination of one or more modified FGFs described herein can be administered.

The compositions described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

In some embodiments, in the case wherein the patient's condition does not improve, upon the doctor's discretion the administration of the compositions are administered chronically, that is, for an extended period of time, including throughout the duration of the patient's life in order to ameliorate or otherwise control or limit the symptoms of the patient's disease or condition.

In some embodiments, in the case wherein the patient's status does improve, upon the doctor's discretion the administration of the compositions is given continuously; alternatively, the dose of drug being administered is temporarily reduced or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The desired dose may conveniently be presented in a single dose or as divided doses administered simultaneously (or over a short period of time) or at appropriate intervals, for example as two, three, four or more sub-doses per day.

In some embodiments, the pharmaceutical composition described herein is in unit dosage forms suitable for single administration of precise dosages. In unit dosage form, the formulation is divided into unit doses containing appropriate quantities of one or more modified FGFs. In some embodiments, the unit dosage is in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection are presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

In some embodiments, a pharmaceutical composition comprising one or more modified FGFs comprises a matrix or network containing one or more of the following: collagen, fibrin, fibrinogen, fibronectin, and/or alginate. For example, a pharmaceutical composition comprising one or more modified FGFs is formulated as a fibrin plug or fibrin glue. In some embodiments, compositions comprising a modified FGF is embedded or soaked into a medical or surgical device, such as a fabric, bandage, suture, sponge, etc. or other polymers, which, in some embodiments, safely degrade over time.

In some embodiments, pharmaceutical compositions described herein comprise one or more modified FGFs described herein conjugated to a dressing, a polymer, or a biological matrix. In various embodiments, the FGF-conjugated dressing, polymer, or matrix is administered to a patient. In various embodiments, the dressing, polymer and/or biological matrix is modified to create thiol functional groups capable of forming disulfide bonds with the modified FGF. In various embodiments, a material used to modify the biological matrix comprises at least one of: thiol-modified collagen and thiol-modified hyaluronic acid. In various embodiments, a material used to create thiol functional groups that form disulfide bonds with the modified FGFs comprise thiol-modified collagen (aka Gelin -S) and thiol modified hyaluronic acid (aka Glycosil) are commercially available from ESI Bio.

In some embodiments, pharmaceutical compositions described herein comprise one or more modified FGFs described herein conjugated to a dressing, a polymer, or a biological matrix. In some embodiments, the modified FGFs described herein are complexed with one or more biological matrices. In some embodiments, the one or more biological matrices comprise a polymer of polylactide, a copolymer of polylactide, and/or a decellularized extracellular matrix. In some embodiments, the one or more biological matrices comprise hydroxyapatite. In various embodiments, the one or more biological matrices comprise a hydroxyapatite nanoparticle. In various embodiments, the one or more biological matrices comprise a sulfhydryl derivatized hydroxyapatite nanoparticle.

In some embodiments, pharmaceutical compositions described herein comprise one or more modified FGFs described herein conjugated to a dressing, a polymer, or a biological matrix. In some embodiments, modified FGFs described herein are administered as a dressing to a wound site by bringing immobilized FGF directly into contact with the wound and releasing the modified FGF into the wound. In some embodiments, the modified FGF is released into the wound over time at one or more doses. In some embodiments, releasing the modified FGF to the wound site using a dressing increases the efficacy of the treatment and decreases the potential for side effects distal to the wound site. In some embodiments, one or more biological matrices to which the FGF is complexed form a part of the regenerated tissue and enhance healing and regeneration of normal structure.

### Combination Treatments

The modified FGFs and pharmaceutical compositions may also be used in combination with other therapeutic agents that are selected for their therapeutic value for the condition to be treated. Such agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the clinician. The initial administration can be made according to established protocols recognized in the field, and then, based upon the observed effects, the dosage, modes of administration and times of administration can be modified by the clinician.

The particular choice of these optional additional agents used will depend upon the diagnosis of the attending physicians and their judgment of the condition of the patient and the appropriate treatment protocol. In some embodiments, the agents are administered concurrently (e.g., simultaneously, essentially simultaneously or within the same treatment protocol) or sequentially, depending upon the nature of the disease, disorder, or condition, the condition of the patient, and the actual choice of agents used. The determination of the order of administration, and the number of repetitions of administration of each therapeutic agent during a treatment protocol, is well within the knowledge of the physician after evaluation of the disease being treated and the condition of the patient.

In some embodiments, the pharmaceutical agents which make up the combination therapy disclosed herein are a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For example, the modified FGF is incorporated into formulations that contain other active ingredients such as steroids, antibiotics, anti-inflammatories, cytokines such as IL-1 or analogs of IL-1, or antagonists of cytokines such as inhibitors of IL-17.

Other exemplary cytokines include, but are not limited to, interleukins (e.g., IL-2, IL-3, IL-4, IL-5, IL-6, IL-7, IL-8, IL-9, IL-10, IL-11, IL-12, IL-13, IL-14 IL-15, IL-16, IL-17, IL-18, IL-la, IL-1(3, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and migration inhibitory factor MIF.

In some embodiments, combinations or pharmaceutical compositions described herein are administered in immunosuppressive therapy to reduce, inhibit, or prevent activity of the immune system. Immunosuppressive therapy is clinically used to: prevent the rejection of transplanted organs and tissues; treatment of autoimmune diseases or diseases that are most likely of autoimmune origin; and treatment of some other non-autoimmune inflammatory diseases.

In some embodiments, the modified FGFs and pharmaceutical compositions described herein are administered with one or more anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, and COX-2 specific inhibitors (such as, but not limited to, celecoxib, rofecoxib, valdecoxib, parecoxib, etoricoxib, lumiracoxib, CS-502, JTE-522, L-745,337 and NS398).

Corticosteroids, include, but are not limited to: betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Other agents used as anti-inflammatories include those disclosed in U.S. patent publication 2005/0227929, herein incorporated by reference.

Some commercially available anti-inflammatories include, but are not limited to: Arthrotec® (diclofenac and misoprostol), Asacol® (5-aminosalicyclic acid), Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer® (aspirin), Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In one embodiment, compositions described herein are administered with leukotriene receptor antagonists including, but are not limited to, BAY u9773 (see EP 00791576; published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

In some embodiments, the modified FGFs and pharmaceutical compositions described herein are administered with one or more Rho kinase inhibitors. In some embodiments, the modified FGFs and pharmaceutical compositions described herein are administered with one or more additional growth factors, including, but not limited to epidermal growth factor (EGF) and nerve growth factor (NGF) (See, e.g., see Joyce et al. Invest Ophthalmol. Vis Sci. 2009; 50:2116-2122), vascular endothelial growth factor (VEGF), transforming growth factor alpha and beta (TGF-alpha and TFG-beta), platelet-derived endothelial growth factor (PD-ECGF), platelet-derived growth factor (PDGF), tumor necrosis factor alpha (TNF-alpha), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxide synthase (NOS).

In some embodiments, the modified FGFs and pharmaceutical compositions described herein are used to treat bone injury, surgical sites, for the fusion of bone such as the fusion of vertebrae in spinal corrective surgery, and/or the healing and regeneration of bone tissue such as in periodontal surgery. In some embodiments, pharmaceutical compositions comprise one or more modified FGFs conjugated to a dressing, a polymer, or a biological matrix. In various embodiments, an FGF-conjugated dressing, polymer, or matrix is administered to a patient. In various embodiments, the dressing, polymer and/or biological matrix is modified to create thiol functional groups capable of forming disulfide bonds with the modified FGF. In various embodiments, a material used to modify the biological matrix comprises at least one of: thiol-modified collagen and thiol-modified hyaluronic acid. In various embodiments, a material used to create thiol functional groups that form disulfide bonds with the modified FGFs comprise thiol-modified collagen (aka Gelin -S) and thiol modified hyaluronic acid (aka Glycosil) are commercially available from ESI Bio.

In some embodiments, the modified FGFs and pharmaceutical compositions described herein are used to treat bone injury, surgical sites, for the fusion of bone such as the fusion of vertebrae in spinal corrective surgery, and/or the healing and regeneration of bone tissue such as in periodontal surgery. In some embodiments, the modified FGFs described herein are complexed with one or more biological matrices. In some embodiments, the one or more biological matrices comprise a polymer of polylactide, a copolymer of polylactide, and/or a decellularized extracellular matrix. In some embodiments, the one or more biological matrices comprise hydroxyapatite. In various embodiments, the one or more biological matrices comprise a hydroxyapatite nanoparticle. In various embodiments, the modified FGFs are prepared as a complex with thiol derivatized hydroxyapatite. The production of thiol functionalized hydroxyapatite is described, as a non-limiting example, by Williams (Williams et al. 2013 J Mater Chem B 1, 4370). In various embodiments, the one or more biological matrices comprise a sulfhydryl derivatized hydroxyapatite nanoparticle.

In some embodiments, the modified FGFs and pharmaceutical compositions described herein are used to treat bone injury, surgical sites, for the fusion of bone such as the fusion of vertebrae in spinal corrective surgery, and/or the healing and regeneration of bone tissue such as in periodontal surgery. In some embodiments, modified FGFs described herein are administered as a dressing to a wound site by bringing immobilized FGF directly into contact with the wound and releasing the modified FGF into the wound. In some embodiments, the modified FGF is released into the wound over time at one or more doses. In some embodiments, releasing the modified FGF to the wound site using a dressing increases the efficacy of the treatment and decreases the potential for side effects distal to the wound site. In some embodiments, one or more biological matrices to which the FGF is complexed form a part of the regenerated tissue and enhance healing and regeneration of normal structure.

In some embodiments, the modified FGFs and pharmaceutical compositions described herein are used to treat soft tissue injury including skin injury. In some embodiments, modified FGFs are prepared as a complex with polymers routinely used in medical devices such as polylactide. The production of thiol derivatized polylactide based polymers is described, as a non-limiting example, by Themistou (Themistou et al 2014 Polym Chem 5:1405). In some embodiments, production of polymers with thiol functional units complexed with modified FGFs described herein is accomplished using methodologies for the formation of disulfides within and between cysteine residues. As a non-limiting example, such methodologies are described in Bradshaw (Bradshaw et al J Biol Chem 1967 242:3789-98).

### Kits/Articles of Manufacture

For use in the therapeutic applications described herein, kits and articles of manufacture are also provided herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of ophthalmic formulations of the modified FGFs and pharmaceutical compositions provided herein are contemplated as are a variety of treatments for any ocular disease, disorder, or condition that would benefit by administration of a modified FGF ore pharmaceutical composition described herein.

For example, the container(s) can include a modified FGF such as a modified FGF having a sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 109, or a combination thereof. The container(s) optionally have a sterile access port. Such kits optionally comprising compounds with an identifying descriptions or labels or instructions relating to their use in the methods described herein.

A kit will typically include one or more additional containers, each with one or more of various materials (such as reagents, optionally in concentrated form, and/or devices) desirable from a commercial and user standpoint for use of a modified FGF described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a modified FGF pharmaceutical composition can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a modified FGF provided herein formulated in a compatible pharmaceutical carrier can also be prepared, placed in an appropriate container, and labeled for treatment of an indicated condition.

### EXAMPLES

These examples are provided for illustrative purposes only and not to limit the scope of the claims provided herein. The starting materials and reagents used in the examples described herein may be synthesized or can be obtained from commercial sources.

### Example 1: Stimulation of Wound Healing Using a Modified FGF

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108.

The modified FGF is produced by chemical synthesis of the gene and insertion into the pET32a expression vector. E. coli containing the expression vector is grown in LB broth, harvested by centrifugation, rinsed in phosphate buffered saline, and lysed by sonication. The lysate is clarified by centrifugation and the expressed protein in the supernatant purified using a Ni column, cleaved using enterokinase, and the resulting modified FGF isolated using a heparin affinity column. The production of proteins including FGFs documented in the following references: Zakrsewska et al., 2009 J Biol Chem 284:25388-25403; as is the purification of FGF via heparin binding Zakrsewska, and also Xiong et al, Methods in Molecular Biology vol 424 vol 1 pp 213, Posch, A., Ed.

The purified FGF is applied at 5-10 μg/cm2 in saline daily to the site of full thickness splinted excisional wounds on the back of db/db mice. These mice have impaired wound healing due to diabetes and are a well-known model of wound healing (Mellin et al 1995 J Invest Dermatol 104:850-855). The treated wounds heal much more rapidly than untreated wounds.

### Example 2: Stimulation of Bone Healing Using Modified FGF-Hydroxyapatite Composition

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108.

The modified FGF is produced as described in example 1. Functionalized hydroxyapatite nanoparticles composed of SiHA modified with MPTS (3-mercaptopropylsilane) is made as described by Williams (Williams et al 2013 J Mater Chem B 2013 1, 4370). The functionalized SiHA are rinsed in PBS and 1 g of this material is mixed with 100 mg of modified FGF. The mixture is agitated using a rocker platform for 48 hours with exposure to air.

The HA-FGF complex is allowed to settle and the supernatant removed. The resulting slurry is rinsed with PBS. Packed slurry (25 ul) is applied directly to a gap between the ends of a surgically stabilized non-union gap of the femur of a mouse (see Garcia et al., J Surg. Res. 2008 June 1; 147(1):84-91). A comparison of the healing of the bones treated with FGF conjugates shows they heal faster and more completely than those without FGF.

### Example 3: Effects of Modified FGF on Human Corneal Endothelial Cell (HCEC) Proliferation

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

### Methods

Primary cultures of HCECs are thawed and expanded in a FNC-coated T75 flask in growth media (OptiMEM with 8% fetal bovine serum (FBS), insulin/transferrin/selenium, 20 μg/ml ascorbic acid, 200 μg/ml calcium chloride, and antibiotic/antimycotic) supplemented with 10 ng/ml FGF-1 having the sequence of SEQ ID NO: 109.

For the proliferation assay, HCEC cells are passaged using accutase, harvested by centrifugation (200 g×12 min), resuspended in growth media without FGF and plated into FNC-coated 24-well plates (3 plates) at a seeding of 25,000 cells per well in 0.5 ml of growth medium.

At 24 hr post plating, the media is removed and replaced with base media (OptiMEM with 0.4% fetal bovine serum (FBS), insulin/transferrin/selenium, 20 μg/ml ascorbic acid, 200 mg/ml calcium chloride, and antibiotic/antimycotic) with indicated additions. For each plate, the conditions for the negative control are media with no additional FBS and the conditions for the positive control is media with a high concentration of FBS. Each modified FGF protein is diluted to 1 ng/uL in base media as a stock solution and then used to generate the cell media at the tested protein concentrations of 0.3 ng/ml, 1.0 ng/ml, 3 ng/ml, and 10 ng/ml. All controls and mutant proteins are tested in quadruplicate.

Cell numbers are counted manually as the number of cells in a 20× field. The same area of the plate is counted each time, with location marked at day 1 using an ink dot on the bottom surface of the plate.

### Results

The adhesion and regular distribution of the cells is checked 24 hours post plating. These experiments will show that one or more modified FGFs stimulates the HCEC to grow with a dose-response behavior with an EC50 in the ng/ml to μg/ml range. These experiments will show that one or more of the modified FGFs are as potent or more potent than wtFGF.

### Example 4: Effects of Modified FGF on Rabbit Corneal Endothelial Cell Proliferation

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

Primary cultures (passage 1) of rabbit corneal endothelial cells are seeded onto 12 well plates in the presence of one or more modified FGFs (long/ml), fetal bovine serum (8% FBS) as a positive control, or no addition of fetal bovine serum as a negative control (−control). Cell numbers are counted under phase contrast for each of 3 wells per treatment. The data shows one or more modified FGFs stimulate rabbit corneal endothelial cell proliferation.

### Example 5: Effects of Modified FGF on Human Corneal Endothelial Cell Proliferation

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

Primary cultures (passage 1) of human corneal endothelial cells from a healthy donor are seeded onto 24 well plates in the presence of fetal bovine serum (FBS, 8%) and 24 hours later treated with the indicated concentrations of one or more modified FGFs in media with low (0.8%) FBS. Cell numbers are counted under phase contrast for each of 4 wells per treatment. Pairwise comparison of 10 ng/ml and 3 ng/ml groups to control (low serum without FGF) at day 2 p<0.05. The 8% FBS group serve as positive control. These experiments are performed in the absence of heparin. The data show one or more modified FGFs stimulate human corneal epithelial cell proliferation and are dose responsive therein.

### Example 6: Determination of Dose-Response Stimulation of CECs

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

FGF-1 is used as the base molecule for generation of modified FGFs because it is unique among FGFs in stimulating all seven FGF receptor isoforms. The FGF-1 protein having the sequence of SEQ ID NO: 109, has been demonstrated to retain activity in a 3T3 cell proliferation assay (Dubey et al. Journal of molecular biology. 2007; 371(1):256-68), and the amino acid substitutions and stabilization of the structure does result in any changes to the FGF receptor stimulation since there are minimal changes to the surface exposed residues.

Dose-response curves are determined for both migration (via the in vitro scratch assay) and mitogenic stimulationof CECs. Primary cultures of rabbit CECs are established using established procedures, e.g., the procedure described by Kay et al. (Kay et al. Investigative ophthalmology & visual science. 1993; 34(3):663-72; Lee et al., Investigative ophthalmology & visual science. 2009; 50(5):2067-76). Briefly, Descemet's membrane complex from rabbit eyes is treated with 0.2% collagenase and 0.05% hyaluronidase for 90 min at 37° C. Dissociated cells are cultured in DMEM supplemented with 15% fetal bovine serum and 50 ug/ml gentamycin. Cells are allowed to grow to confluence and are subcultured using trypsin/EDTA. First passage cells are used for all experiments. The scratch assay is performed by allowing first passage cells to grow to confluence in 6-well plates and using a rubber policeman to create a scratch in the monolayer. Proliferation assays are performed in 12-well plates using, e.g., a Click-IT assay kit (Life Technologies). Dose response curves are generated for FGF-1 in the presence and absence of heparin. FGF-1 in the presence of heparin and FGF-2 are used as positive controls.

The doses proposed in the in vivo experiments are based on the assumption that the ED₅₀ for one or more modified FGFs is similar to the ED₅₀ for the wild type FGF-1. The determined ED₅₀ are used to adjust the dose for the in vivo experiments. The results indicate one or more modified FGFs stimulates migration and/or proliferation of the rabbit CECs.

### Example 7: Demonstration that One or More Modified FGFs Localize to Areas of Corneal Injury or CEC Progenitors in Whole Cornea (Organ Culture) and In Vivo Via Anterior Chamber (Intracameral) Injection

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

Effective therapy requires that the drug get to the target tissue and be present for long enough to act. Wild-type FGF-1 (wtFGF-1) requires a 3× mass excess of heparin for stability and the heparin in the formulation can interfere with the targeting of the FGF to the wound site in topical ophthalmic applications. Pharmacokinetic studies comparing wtFGF-1 with and without heparin to one or more modified FGFs confirm that the one or more modified FGFs partitions into the tissue compartment more effectively than wtFGF-1 and is released slowly from that compartment (Xia et al. PloS one. 2012; 7(11):e48210). Corneal injury activates stromal keratocytes to produce heparins (Brown et al., Journal of cellular biochemistry. 1995; 59(1):57-68).

The combination of the ability to administer one or more modified FGFs without heparin and the low protein concentration of the aqueous humor results in one or more modified FGFs localizing to the endothelial surface and to exposed stroma in areas of lesion.

Labeled one or more modified FGFs is used to demonstrate one or more modified FGFs is bound to the tissue compartment and releases slowly, first in whole organ cultured corneas and then in vivo following intracameral administration.

One or more modified FGFs is labeled with dye as described in the art (Xu, et al., The Journal of biological chemistry. 2012; 287(47):40061-73). Briefly, in this procedure one or more modified FGFs is complexed with heparin before dye derivatization so that the dye is not bound to the heparin binding site and therefore derivatization does not disrupt heparin binding. It is still possible that the dye may bind to receptor binding areas of the protein, but the initial interactions with tissue are predominantly via proteoglycans rather than the FGF receptors, and the amount of one or more modified FGFs bound to receptors is not detectable in this assay.

To model intracameral injection of one or more modified FGFs using corneal organ culture, explanted corneas including the peripheral cornea are incubated with one or more labeled modified FGFs for 30 minutes followed by rinsing for varying periods of time in media without the one or more labeled modified FGFs. Corneas are scratched to create an endothelial lesion across the central cornea. Corneas are used immediately or are incubated in culture media for 24 hours after scratching to allow expression of heparans by the stromal cells before assaying modified FGF binding. To evaluate the dependence of binding on heparins, corneas are exposed to one or more labeled modified FGFs in the absence or presence of heparin (10 ug/ml).

### Example 8: Acceleration of Corneal Healing in an In Vivo Animal Model

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF is produced as described in example 1.

This study is directed to semi-quantitative in vivo PK measurements. For in vivo experiments, one or more labeled modified FGFs is administered by intracameral injection via the peripheral cornea in the absence of heparin, and the fluorescently labeled protein followed by periodic inspection via confocal fluorescence microscopy with Heidelberg Retinal Tomography (HRT)+Rostock Cornea Module. Injected eyes are evaluated at 30 min, 2 hours, 8 hours, 24 hours and 48 hours post injection. If label is still present at 48 hours, animals are followed for up to 2 weeks.

The administration paradigm in the in vivo experiment assumes that the binding of the one or more modified FGFs to the cornea can be measured and that the time course of disappearance of the bound one or more modified FGFs is at least as long as that expected by dilution of the aqueous (t_{1/2} of about 90 minutes). In the event that the disappearance of the one or more modified FGFs follows a more rapid course, the one or more modified FGFs is administered more frequently in the animal model.

Once one or more modified FGFs is confirmed to stimulate the CECs and an EC₅₀ established, the effect of the one or more modified FGFs in ᵥᵢᵥₒ is measured using the rabbit transcorneal freeze damage model of corneal endothelial damage (Okumura et al. The British journal of ophthalmology. 2011; 95(7):1006-9). Briefly, a 7 mm steel probe is frozen in liquid nitrogen and then held to the cornea of the rabbit for 15 s. This freezes the cornea through to the endothelial cell layer and generates a reproducible region of CEC loss, with corneal edema and a cell free area of the inner corneal surface 24 hours later.

Rabbits are subjected to transcorneal freezing under general anesthesia. The contralateral eye serves as a control. Once the ice ball in the anterior chamber has melted, the lesions are imaged microscopically and photographed to verify appropriate lesion generation and size. One or more modified FGFs are administered intracamerally via a 30 ga needle into the aqueous via the peripheral cornea. Injection occurs across an area of cornea distant from the lesion. Corneas are examined microscopically and photographed at 24 and 48 hrs, including OCT imaging of corneal thickness to evaluate damage to the stroma as well as confocal microscopy with the HRT and CEC counting. At 48 hours, the animals are sacrificed and corneas are harvested and are stained with alizarin red. The area of the endothelial lesions is quantitated using image analysis.

One or more modified FGFs is administered intracamerally starting at a dose of 100 ng (10 ul of a 10 ug/ml solution) in saline approximately 60 min after lesioning. A 100 ng dose into the aqueous of a rabbit gives an initial concentration of approximately 400 ng/ml and given the turnover of aqueous humor of 2-3 ul/min the concentration of one or more modified FGFs in the aqueous falls below 2 ng/ml (the ED₅₀ in the 3T3 cell assay (Dubey et al. Journal of molecular biology. 2007; 371(1):256-68)) at 11 hours post dose. On this basis, only a single dose of the one or more modified FGFs is administered for these experiments, and the dosing frequency is adjusted if no effect is seen at this dose. Agonists such as FGF-1 often exhibit a bell shaped dose response curve; therefore, other groups of animals are treated with lower or higher doses to establish a dose response curve.

In the rabbit model, administration of the Rho kinase (ROCK) inhibitor Y-27632 reduces the area of lesion to 20% of control at 48 hours post lesion. In in vitro models, FGF-2 alone produces a larger effect on migration of endothelial cells than Y-27632 and the two are synergistic (Lee et al. Investigative ophthalmology & visual science. 2006; 47(4):1376-86). If one or more modified FGFs produces an effect at least similar in size to the ROCK inhibitor, six animals per dose group provides a statistically significant result.

These experiments demonstrate that one or more modified FGFs are a potential treatment for FD and other endothelial dystrophies and lays the groundwork for phase II, in which these experiments are repeated with human corneal endothelial cells in vitro and primates in vivo. This efficacy data is sufficient to support an IND filing for the use of one or more modified FGFs in patients that have dystrophy sufficient to be considered for corneal transplants.

### Example 9: Use of a Modified FGF in Patients Undergoing Photorefractive Keratotomy (PRK)

For the following example, one or more of the modified FGFs described herein is used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF will be produced as described in example 1.

Patients who have undergone PRK are treated with a suitable ophthalmic formulation (e.g., a topical solution of 1 mg/ml (0.1%) one or more modified FGFs as an eye drop formulated in phosphate buffered saline). The eye drop is administered daily, e.g., three to four times a day for 7 days. These treated patients experience reepithelialization of the cornea more quickly than untreated patients. Reepithelialization of the cornea is observed fluorescein or lissamine green staining as well as biomicroscopy.

### Example 10: Use of a Modified FGF in Patients with Corneal Endothelial Dystrophy

For the following example, one or more of the modified FGFs described herein will be used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF will be produced as described in example 1.

Patients who have corneal endothelial dystrophy (e.g., Fuch's dystrophy) are treated with a suitable ophthalmic formulation (e.g., a solution of 1 mg/ml (0.1%) one or more modified FGFs formulated in phosphate buffered saline). The FGF solution is administered via intracameral injection every 1-3 months. These treated patients experience increase in the density of the endothelial cells in the central cornea more quickly than untreated patients. The increase in corneal endothelial cell density is viewed using specular microscopy.

### Example 11. Use of Modified FGF in Patients Undergoing Trabeculectomy

For the following example, one or more of the modified FGFs described herein will be used. As a non-limiting example, one or more modified FGFs in this example comprises the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 102, SEQ ID NO: 103, SEQ ID NO: 104, SEQ ID NO: 105, SEQ ID NO: 106, SEQ ID NO: 107, or SEQ ID NO: 108. The modified FGF will be produced as described in example 1.

Patients undergoing trabeculectomy are treated with one or more modified FGFs in a suitable ophthalmic formulation (e.g., a solution of 1 mg/ml (0.1%) of a modified FGF formulated in phosphate buffered saline or as a sustained release formulation applied to the trabeculectomy site). These patients experience more rapid healing of the trabeculectomy site with reduced fibrosis and reduced fibrotic obstruction of the trabeculectomy canal.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following embodiments define the scope of the invention and that methods and structures within the scope of these embodiments and their equivalents be covered thereby.

1. A composition comprising a modified FGF, the modified FGF comprising one or more mutations of human wild type FGF-1 (SEQ ID NO: 109), wherein: said one or more mutated residues are at least one of: at positions K9, K10, P11, K12, C16, C83, C117, P134, P136, and V137 of SEQ ID NO: 109; and wherein: the modified FGF-1 comprises an N-terminal truncation comprising one or more of the first 12 residues; and/or the modified FGF-1 comprises a C-terminal truncation comprising one or more of the last 5 residues.

2-4. (canceled)

5. The composition of claim 1, wherein the modified FGF is truncated up to the first mutated residue in SEQ ID NO: 109.

6. The composition of claim 1, wherein the modified FGF is truncated after the last mutated residue in SEQ ID NO: 109.

7. The composition of claim 1, wherein the modified FGF comprises at least one of the mutations: K12V, C117V, and P134V.

8. The composition of claim 1, wherein the modified FGF comprises at least one of the mutations: K12I, C117I, and P134I.

9. The composition of claim 1, wherein the modified FGF further comprises at least one of the mutations: C16A and C83A.

10. The composition of claim 1, wherein the modified FGF further comprises at least one of the mutations: K9C, K10C, and P11C.

11. The composition of claim 1, wherein the modified FGF further comprises at least one of the mutations: P136C and V137C.

12. The composition of claim 1, wherein mutating one or more of residues 9, 10 and 11 to cysteine and mutating one or more of residues 136 and 137 to cysteine introduces a stabilizing disulfide bond between the mutated residues.

13. The composition of claim 1, wherein the modified FGF 1 comprises a truncated form of a sequence selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, or SEQ ID NO: 108.

14. The composition of claim 1, wherein the modified FGF is conjugated to a dressing, a polymer, or a biological matrix.

15. The composition of claim 14, wherein the biological matrix comprises at least one of sulfhydryl derivatized hydroxyapatite, a polymer or copolymer of polylactide, and a decellularized extracellular matrix.

16. The composition of claim 14, wherein the modified FGF is conjugated to a modified nanoparticle; or a sulfhydryl derivatized hydroxyapatite nanoparticle.

17. (canceled)

18. The composition of claim 16, wherein the biological matrix is modified to create thiol functional groups capable of forming disulfide bonds with the modified FGF.

19. The composition of claim 18, wherein a material used to modify the biological matrix comprises at least one of: thiol-modified collagen and thiol-modified hyaluronic acid.

20. A method of treating an injury, disease, or condition in a mammal comprising administering to the mammal a pharmaceutical composition comprising:
a) a modified FGF comprising one or more mutations of human wild type FGF-1 (SEQ ID NO: 109) at positions K9, K10, P11, K12, C16, C83, C117, P134, P136, and V137; and
b) a pharmaceutically acceptable carrier, excipient, or diluent.

21. The method of claim 20, wherein treating comprises treating or preventing an ocular disease disorder or condition in a mammal.

22. The method of claim 20, wherein treating comprises treating a corneal or retinal disease in a mammal.

23.-26. (canceled)

27. The method of claim 20, wherein the modified FGF comprises an N-terminal truncation comprising one or more of the first 12 residues.

28. The method of claim 20, wherein the modified FGF comprises a C-terminal truncation comprising one or more of the last 5 residues.

29.-120. (canceled)


SEQUENCE LISTING (110 sequences)

SEQ ID NO: 1 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 2 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 3 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 4 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 5 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 6 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 7 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 8 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 9 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 10 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 11 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 12 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 13 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 14 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 15 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 16 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 17 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 18 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 19 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 20 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 21 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 22 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 23 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 24 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 25 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 26 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 27 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 28 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 29 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 30 - protein, 140 aa, Artificial Sequence
  Description of Artificial Sequence Synthetic polypeptide
FNLPPGNYCKPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 31 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 32 - protein, 140 aa, Artificial Sequence
  Description of Artificial Sequence Synthetic polypeptide
FNLPPGNYKCPVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 33 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 34 - protein, 140 aa, Artificial Sequence
  Description of Artificial Sequence Synthetic polypeptide
FNLPPGNYKKCVLLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 35 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 36 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 37 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 38 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPVSSD

SEQ ID NO: 39 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 40 - protein, 140 aa, Artificial Sequence
  Description of Artificial Sequence Synthetic polypeptide
FNLPPGNYKKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 41 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 42 - protein, 140 aa, Artificial Sequence
  Description of Artificial Sequence Synthetic polypeptide
FNLPPGNYCKPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 43 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 44 - protein, 140 aa, Artificial Sequence
  Description of Artificial Sequence Synthetic polypeptide
FNLPPGNYKCPVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 45 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLCVSSD

SEQ ID NO: 46 - protein, 140 aa, Artificial Sequence
  Description of Artificial Sequence Synthetic polypeptide
FNLPPGNYKKCVLLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSVKRGPRTHYGQKAILFLVLPCSSD

SEQ ID NO: 47 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 48 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 49 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 50 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 51 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 52 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 53 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 54 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 55 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 56 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 57 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 58 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 59 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 60 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPVSSD

SEQ ID NO: 61 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 62 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 63 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 64 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 65 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 66 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 67 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 68 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 69 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 70 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 71 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 72 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 73 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 74 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 75 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 76 - protein, 140 aa, Artificial Sequence
  Description of Artificial Sequence Synthetic polypeptide
FNLPPGNYCKPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 77 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 78 - protein, 140 aa, Artificial Sequence
  Description of Artificial Sequence Synthetic polypeptide
FNLPPGNYKCPILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 79 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 80 - protein, 140 aa, Artificial Sequence
  Description of Artificial Sequence Synthetic polypeptide
FNLPPGNYKKCILLYCSNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 81 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 82 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 83 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 84 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPVSSD

SEQ ID NO: 85 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 86 - protein, 140 aa, Artificial Sequence
  Description of Artificial Sequence Synthetic polypeptide
FNLPPGNYKKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 87 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 88 - protein, 140 aa, Artificial Sequence
  Description of Artificial Sequence Synthetic polypeptide
FNLPPGNYCKPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 89 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 90 - protein, 140 aa, Artificial Sequence
  Description of Artificial Sequence Synthetic polypeptide
FNLPPGNYKCPILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 91 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILCVSSD

SEQ ID NO: 92 - protein, 140 aa, Artificial Sequence
  Description of Artificial Sequence Synthetic polypeptide
FNLPPGNYKKCILLYASNGGHFLRILPDGTVDGTRDRSDQHIQLQLSAESVGEVYIKSTETGQYLAMDTDGLLYGSQTPNEEALFLERLEENHYNTYISKKHAEKNWFVGLKKNGSIKRGPRTHYGQKAILFLILPCSSD

SEQ ID NO: 93 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLCVSSD

SEQ ID NO: 94 - protein, 140 aa, Artificial Sequence
  Description of Artificial Sequence Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPCSSD

SEQ ID NO: 95 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILCVSSD

SEQ ID NO: 96 - protein, 140 aa, Artificial Sequence
  Description of Artificial Sequence Synthetic polypeptide
FNLPPGNYKKPILLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSIKRGPRTHYGQKAILFLILPCSSD

SEQ ID NO: 97 - protein, 140 aa, Artificial Sequence; identical to SEQ ID NO: 41
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 98 - protein, 140 aa, Artificial Sequence; identical to SEQ ID NO: 42
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 99 - protein, 140 aa, Artificial Sequence; identical to SEQ ID NO: 43
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 100 - protein, 140 aa, Artificial Sequence; identical to SEQ ID NO: 44
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 101 - protein, 140 aa, Artificial Sequence; identical to SEQ ID NO: 45
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 102 - protein, 140 aa, Artificial Sequence; identical to SEQ ID NO: 46
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 103 - protein, 140 aa, Artificial Sequence; identical to SEQ ID NO: 87
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 104 - protein, 140 aa, Artificial Sequence; identical to SEQ ID NO: 88
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 105 - protein, 140 aa, Artificial Sequence; identical to SEQ ID NO: 89
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 106 - protein, 140 aa, Artificial Sequence; identical to SEQ ID NO: 90
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 107 - protein, 140 aa, Artificial Sequence; identical to SEQ ID NO: 91
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 108 - protein, 140 aa, Artificial Sequence; identical to SEQ ID NO: 92
  Description of Artificial Sequence Synthetic polypeptide
  MISC_FEATURE (1)..(5): This region may or may not be present in its entirety
  MISC_FEATURE (138)..(140): This region may or may not be present in its entirety

SEQ ID NO: 109 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 110 - protein, 5 aa, Artificial Sequence
  Description of Artificial Sequence Synthetic peptide
DDDDK